(19) United States
(12) Patent Application Publication
(10) Pub. No.: US 2011/0251390 A1
(43) Pub. Date: Oct. 13, 2011

(54) NICOTINE-BASED COMPOUNDS USEFUL FOR ASYMMETRIC SYNTHESIS

(76) Inventors: Daniel L. Comins, Cary, NC (US); Sonja S. Capracotta, Canton, MI (US)

(21) Appl. No.: 13/122,060

(22) PCT Filed: Oct. 2, 2009
(86) PCT No.: PCT/US09/59390

§ 371 Date: Jun. 10, 2011

Related U.S. Application Data
(60) Provisional application No. 61/102,516, filed on Oct. 3, 2008.

(51) Int. Cl. C07F 3/06; C07C 33/28; C07F 9/58; C07C 33/30; C07D 401/14; C07C 33/20; C07C 33/46; C07C 43/23; C07D 401/04; C07C 67/00

(52) U.S. Cl. 546/2; 546/276.4; 568/807; 546/21; 560/81; 546/256; 568/715; 568/812; 568/648; 568/813

(57) ABSTRACT

Chiral amino alcohol and amino phosphine compounds are provided herein, which compounds are useful for asymmetric synthesis.

### RELATED APPLICATIONS

This application claims priority to U.S. Provisional Application No. 61/102,516, filed Oct. 3, 2009, the disclosure of which is incorporated by reference herein in its entirety.

### FIELD OF THE INVENTION

The invention relates to chiral compounds and the use thereof in asymmetric reactions.

### BACKGROUND

Many enantiomerically pure products with biological activities are commonly used in the pharmaceutical industry due to their effectiveness. As such, procedures that produce stereochemically pure or substantially pure compounds are highly desirable.

Asymmetric synthesis using chiral compounds complexed to a transition metal has become the preferred way to make enantiopure material. For example, selectively synthesizing secondary alcohols that are ubiquitous in natural products and pharmaceuticals can be achieved through the catalytic asymmetric addition of an alkylzinc to an aldehyde.

Chiral molecules to be used in these reactions can be found in nature. However, this “chiral pool” of enantiopure compounds is limited. In addition, methods for extracting these molecules from plants and other sources are generally inefficient and costly.

Therefore, new molecules useful in asymmetric synthesis are needed.

### SUMMARY

Chiral amino alcohols and amino phosphines are provided herein, which are useful for, inter alia, complexing to metals to form catalysts for asymmetric synthesis (e.g., for the preparation of optically active compounds for the pharmaceutical, agrochemical, fragrances and flavors industries).

Provided herein are amino alcohol compounds of Formula I:

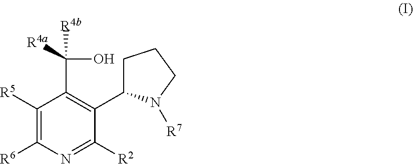

wherein:
R⁷ is H or alkyl;
R², R⁵ and R⁶ are each independently H, alkyl, halo, alkoxy, aryl or heteroaryl; and
R⁴ and R^{4b} are each independently H, alkyl, aryl or heteroaryl.

Also provided herein are amino alcohol compounds of Formula II:

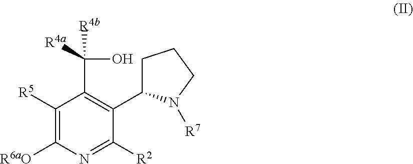

wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H, alkyl, aryl or heteroaryl.

Further provided are amino alcohol compounds of Formula III:

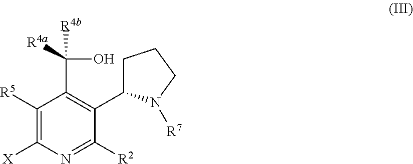

wherein:
R⁷ is H or alkyl;
R² and R⁵ are each independently H, alkyl, halo, alkoxy, aryl or heteroaryl;
X is halo; and
R^{4a} and R^{4b} are each independently H, alkyl, aryl or heteroaryl.

Also provided are amino alcohol compounds of Formula (III)(A)(3):

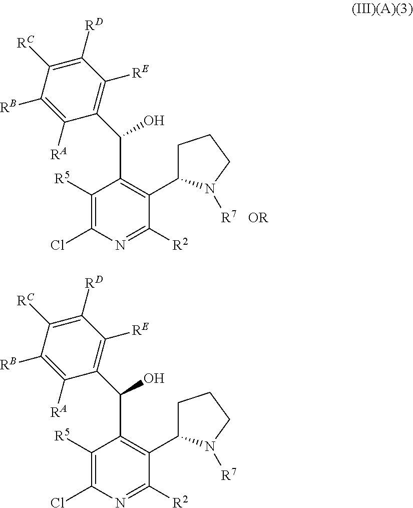

wherein:
R⁷ is H or alkyl;
R² and R⁵ are each independently H, alkyl, halo, alkoxy, aryl or heteroaryl; and
R^{A}, R^{B}, R^{C}, R^{D} and R^{E} are each independently halo (e.g., fluoro).

Also provided are amino alcohol compounds of Formula (III)(B)(4):

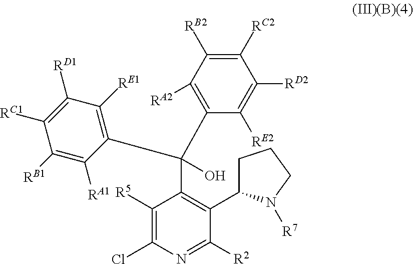

wherein:
R⁷ is H or alkyl;
R² and R⁵ are each independently H, alkyl, halo, alkoxy, aryl or heteroaryl; and
R^{A1}, R^{B1}, R^{C1}, R^{D1}, R^{E1}, R^{A2}, R^{B2}, R^{C2}, R^{D2} and R^{E2} are each independently halo (e.g., fluoro).

Also provided are amino alcohol compounds of Formula IV:

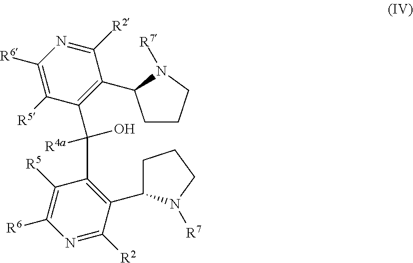

wherein:
R⁷ is H or alkyl;
R², R⁵ and R⁶ are each independently H, alkyl, halo, alkoxy, aryl or heteroaryl R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

Further provided is an amino alcohol compound described herein for use in a catalytic asymmetric addition of a dialkylzinc to an aldehyde (i.e., In a method of catalyzing the asymmetric addition of dialkyl zinc to an aldehyde, the improvement comprising utilizing an amino alcohol compound described herein as a catalyst).

Also provided is an amino alcohol compound as described herein covalently bound to a solid support.

Further provided is a complex comprising a metal (e.g., zinc) and at least one amino alcohol compound described herein.

Also provided herein are phosphine compounds of Formula X:

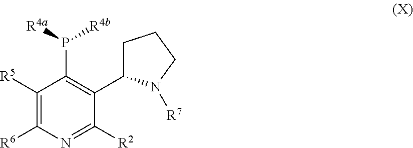

wherein:
R⁷ is H or alkyl;
R², R⁵ and R⁶ are each independently H, alkyl, halo, alkoxy, aryl or heteroaryl; and
R^{4a} and R^{4b} are each independently H, alkyl, aryl or heteroaryl.

Further provided herein are phosphine compounds of Formula XI:

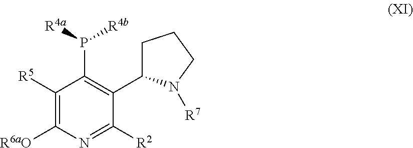

wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H, alkyl, aryl or heteroaryl.

Further provided herein are phosphine compounds of Formula XII:

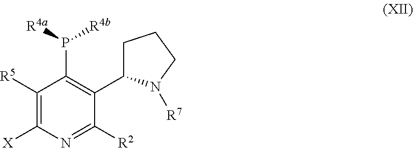

wherein:
R⁷ is H or alkyl;
R² and R⁵ are each independently H, alkyl, halo, alkoxy, aryl or heteroaryl;
X is halo; and
R^{4a} and R^{4b} are each independently H, alkyl, aryl or heteroaryl.

Further provided are phosphine compounds of Formula XIII:

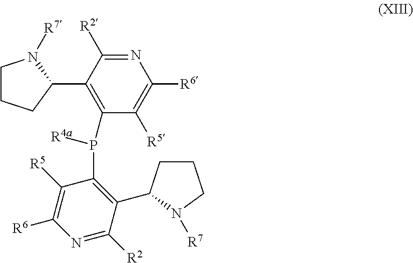

wherein:
R⁷ is H or alkyl;
R², R⁵ and R⁶ are each independently H, alkyl, halo, alkoxy, aryl or heteroaryl R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

Also provided is a phosphine compound described herein for use in a palladium-catalyzed allylic alkylation (i.e., In a method of palladium-catalyzed allylic alkylation, the improvement comprising utilizing a phosphine compound as described herein as a catalyst).

Also provided is a phosphine compound as described herein covalently bound to a solid support.

Further provided is a complex comprising a metal (e.g., zinc, copper, etc.) and at least one phosphine compound described herein.

### BRIEF DESCRIPTION OF THE DRAWINGS

FIG. 1. ORTEP drawing of 73a showing naming and numbering scheme. Ellipsoids are at the 50% probability level, and hydrogen atoms are drawn with arbitrary radii for clarity.

FIG. 2. Spartan molecular model of 77 showing the lowest energy conformation at 35.1 kcal/mol.

FIG. 3. Secondary amino alcohols 73b, 74b, 73c, 74c, 73d, 74d and 77.

FIG. 4. Tertiary amino alcohols 71, 75a, 75b, 75c and 75d.

FIG. 5. Phosphines 97, 98, 99, 100 and 101.

FIG. 6. ORTEP drawing of 115 molecule showing naming and numbering scheme. Ellipsoids are at the 50% probability level, and hydrogen atoms on chiral carbons were drawn with arbitrary radii while the remaining hydrogens were omitted for clarity.

### DETAILED DESCRIPTION OF THE PREFERRED EMBODIMENTS

All patent references are hereby incorporated by reference in their entirety as if set forth fully herein.

### A. Definitions

Elemental and group abbreviations used herein are consistent with their general use in the art unless otherwise specified. For example, “H” is a hydrogen atom; “C” is a carbon atom; “N” is a nitrogen atom; “O” is an oxygen atom; “P” is a phosphorus atom; “hydroxy” is an —OH moiety; “Br” is a bromine atom; “Cl” is a chlorine atom; “I” is an iodine atom; “F” is a fluorine atom; “Ph” is a phenyl group; “Me” is a methyl group; and “Et” is an ethyl group.

“Alcohol” is a compound having, a hydroxyl group (—OH) bound to a carbon atom of an alkyl or substituted alkyl.

“Amine” or “amino” is a compound having a nitrogen atom bound to one or more carbon atoms. Amines include the group —NH₂ as well as —NH₂ groups wherein one or both of the hydrogens is replaced by a suitable substituent as described herein, such as alkyl, alkenyl, alkynyl, cycloalkyl, heterocyclo, aryl, heteroaryl, alkoxy, carbonyl, carboxy, O, N, etc. For example, for a “nitro” group: NO₂, both of the hydrogens are replace by O. Substituted amines may have substituents that are bridging, i.e., form a heterocyclic ring structure that includes the nitrogen atom.

“Phosphine” is a compound having a phosphorus atom bound to one or more carbon atoms. Phosphines include the group —PH₂ as well as —PH₂ groups wherein one or two of the hydrogens is replaced by a suitable substituent as described herein, such as alkyl, alkenyl, alkynyl, cycloalkyl, heterocyclo, aryl, heteroaryl, alkoxy, carbonyl, carboxy, O, N, etc. Substituted phosphines may have substituents that are bridging, i.e., form a heterocyclic ring structure that includes the phosphorus atom.

“Acyl group” is intended to mean a group —C(O)—R, where R is a suitable substituent (for example, an acetyl group, a propionyl group, a butyroyl group, a benzoyl group, or an alkylbenzoyl group).

“Alkyl,” as used herein, refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

“Alkenyl,” as used herein, refers to a straight or branched chain hydrocarbon containing from 1 or 2 to 10 or 20 or more carbons, and containing at least one carbon-carbon double bond, formed structurally, for example, by the replacement of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like.

“Alkynyl,” as used herein, refers to a straight or branched chain hydrocarbon group containing from 1 or 2 to 10 or 20 or more carbon atoms, and containing at least one carbon-carbon triple bond. Representative examples of alkynyl include, but are not limited, to acetylenyl, 1-propynyl, 2-propynyl, 3-butynyl, 2-pentynyl, 1-butynyl and the like.

“Halo” is fluoro, chloro, bromo or iodo.

“Cycloalkyl” refers to a saturated cyclic hydrocarbon group containing from 3 to 8 carbons or more. Representative examples of cycloalkyl include, cyclopropyl, cyclobutyl, cyclopentyl, cyclohexyl, cycloheptyl, and cyclooctyl.

“Heterocyclo,” as used herein, refers to a monocyclic or a bicyclic ring system. Monocyclic heterocycle ring systems are exemplified by any 5 or 6 member ring containing 1, 2, 3, or 4 heteroatoms independently selected from the group consisting of: O, N, and S. The 5 member ring has from 0 to 2 double bonds, and the 6 member ring has from 0 to 3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, sulfoxide, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like.

“Aryl” as used herein refers to a fused ring system having one or more aromatic rings, wherein each of the ring atoms are carbon. Representative examples of aryl include, azulenyl, indanyl, indenyl, naphthyl, phenyl, tetrahydronaphthyl, and the like. The aryl group can be unsubstituted or substituted with from 1 to 10 suitable substituents, as chemically feasible. For example, one or more of the hydrogens bonded to the carbon ring atoms may be substituted with an electron-withdrawing group (e.g., fluoro), an alkyl (e.g., tert-butyl), etc.

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 10 suitable substituents, as chemically feasible. For example, the heteroatom S may be substituted with one or two oxo groups, which may be shown as ═O.

“Alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like.

A “thiol” or “mercapto” refers to an —SH group or to its tautomer ═S.

A “sulfone” as used herein refers to a sulfonyl functional croup, generally depicted as:

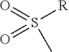

wherein, R can be any covalently-linked atom or atoms, for example, H, halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

A “sulfoxide” as used herein refers to a sulfinyl functional group, generally depicted as:

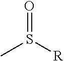

wherein, R can be any covalently-linked atom or atoms, for example, H. halo, hydroxy, acyl, alkyl, alkenyl, alkynyl, cycloalkyl, heterocyclo, aryl, heteroaryl, alkoxy, amino, amide, thiol, sulfone, sulfoxide, oxo, oxy, nitro, carbonyl, carboxy, amino acid sidechain, amino acid and peptide.

“Carbonyl” is a functional group having a carbon atom double-bonded to an oxygen atom (—C═O). “Carboxy” as used herein refers to a —COOH functional group, also written as —(C═O)—OH.

The term “electron-withdrawing group” is an atom, group or moiety that draws electrons to itself more than a hydrogen atom would at the same position. Exemplary electron-withdrawing groups include nitro, ketone, aldehyde, sulfonyl, trifluoromethyl, —CN, halo, acid, ester, amide and the like.

The term “electron-donating group” is an atom, group or moiety that draws electrons to itself less than a hydrogen atom would at the same position. Exemplary electron-donating groups include amino, methoxy, and the like.

The term “chiral” refers to molecules which have the property of non-superimposability of their mirror image partner. The term “stereoisomers” refers to compounds which have identical chemical constitution, but differ with regard to the arrangement of the atoms or groups in space. In particular, “enantiomers” refer to two stereoisomers of a compound which are non-superimposable mirror images of one another. “Diastereomers,” on the other hand, refers to stereoisomers with two or more centers of dissymmetry and whose molecules are not mirror images of one another.

“Metal” as used herein refers to a metal atom capable of coordinating or complexing to one or more ligands as described herein to form a catalyst useful for, inter alia, asymmetric catalysis. Examples of metals useful in such catalysts include, but are not limited to, Zn, Cu, Pd, Mn, Rh, Pt, Ir, Ni, Ti and the like.

### B. Compounds

Compounds are provided below, which compounds are useful, inter alia, as ligands for complexing to metals to form catalysts for asymmetric synthesis (e.g., for the preparation of optically active compounds (e.g., enantiomers) for the pharmaceutical, agrochemical, fragrances and flavors industries). Also provided are catalyst complexes, which complexes include one or more of the compounds described herein complexed to a metal or metals. In some embodiments, ligands are monodentate. In some embodiments, bidentate. In some embodiments, ligands are tridentate. In some embodiments, ligands are tetradentate.

According to some embodiments, compounds disclosed herein are useful for enantioselective, diastereoselective, and/or regioselective reactions. An enantioselective reaction is a reaction which converts an achiral reactant to a chiral product enriched in one enantiomer. Enantioselectivity is generally quantified in the art as “enantiomeric excess” (ee), which is defined as follows:

% enantiomeric excess A(ee)=(% enantiomer A)−(% enantiomer B)

where A and B are the enantiomers formed. An enantioselective reaction yields a product with an ee greater than zero. Preferred enantioselective reactions yield a product with an ee greater than 20%, more preferably greater than 50%, even more preferably greater than 70%, and most preferably greater than 80%.

Additional terms that are used in conjunction with enatioselectivity include “optical purity” or “optical activity.” When present in a symmetric environment, enantiomers have identical chemical and physical properties except for their ability to rotate plane-polarized light by equal amounts but in opposite directions.

In some embodiments, compounds of the formulas disclosed herein contain chiral centers, e.g., asymmetric carbon atoms. Unless a certain configuration is specified by the formula, all stereoisomers of the formula are included at each chiral center.

Geometric isomers of double bonds and the like may also be present in the compounds disclosed herein, and all such stable isomers are included within the present invention unless otherwise specified. Also included in the compounds described herein are tautomers and rotamers.

In some embodiments, compounds disclosed herein are derivatives of nicotine (e.g., S-nicotine, R-nicotine, or a racemic mixture). In some embodiments, compounds are enantiomerically pure or substantially enantiomerically pure at one or more specified positions in the formula (e.g., between 50, 60, 70, 80, 90 or 95% or more and 100% of a certain enantiomer or other stereoisomer specified as present in the formula, versus all other sterioisomers at that position). In other embodiments, one or more of the enantiomers or diastereomers are present among other compounds, e.g., in a mixture (for example, a racemic mixture). If desired, the resolution of stereoisomers or of racemates into enantiomeric forms may be performed in accordance with procedures described herein and known in the art.

Compounds and complexes described herein can be prepared as detailed below or in accordance with known procedures, or variations of the same that will be apparent to those skilled in the art. See, e.g., U.S. Patent Publication No. 2007/0232665 and U.S. Pat. Nos. 7,067,672 and 7,179,917 to Comins et al., which are incorporated by reference herein in their entireties.

In some embodiments, compounds and/or complexes described herein are covalently bound to insoluble supports. For example, compounds may be attached at the 2, 6, 5, or 1′ position of the nicotine structure, or through groups connected to these positions:

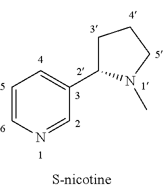

### 1. Amino Alcohol Compounds

Provided herein are amino alcohol compounds of Formula I:

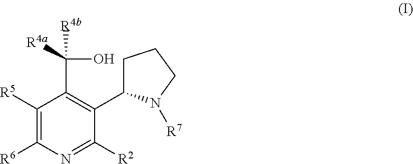

wherein:
R⁷ is H or alkyl;
R², R⁵ and R⁶ are each independently H, alkyl, halo, alkoxy, aryl or heteroaryl; and
R^{4a} and R^{4b} are each independently H, alkyl, aryl or heteroaryl.

Also provided herein are amino alcohol compounds of Formula III:

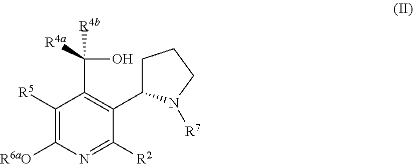

wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H, alkyl, aryl or heteroaryl.

Also provided herein are amino alcohol compounds of Formula III:

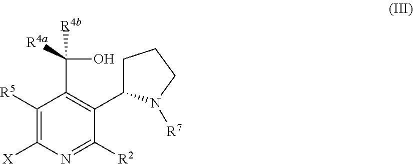

wherein:
R⁷ is H or alkyl;
R² and R⁵ are each independently H, alkyl, halo, alkoxy, aryl or heteroaryl;
X is halo; and
R^{4a} and R^{4b} are each independently H, alkyl, aryl or heteroaryl.

In some embodiments of Formula III, X is Cl; and one of either R^{4a} or R^{4b} is H, and the other is aryl; examples of which include, but are not limited to, Formula (III)(A)(1), Formula (III)(A)(2) and Formula (III)(A)(3):

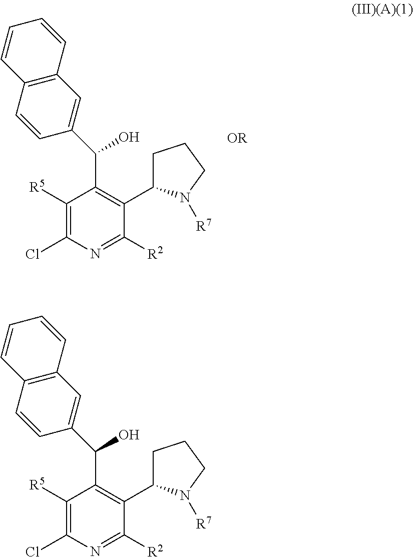

wherein:
R⁷ is H or alkyl; and
R² and R⁵ are each independently H, alkyl, halo, alkoxy, aryl or heteroaryl.

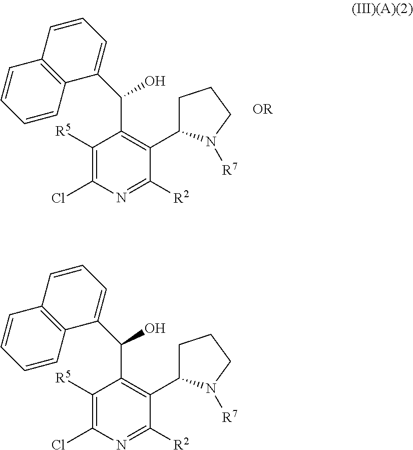

wherein:
R⁷ is H or alkyl; and
R² and R⁵ are each independently H, alkyl, halo, alkoxy, aryl or heteroaryl.

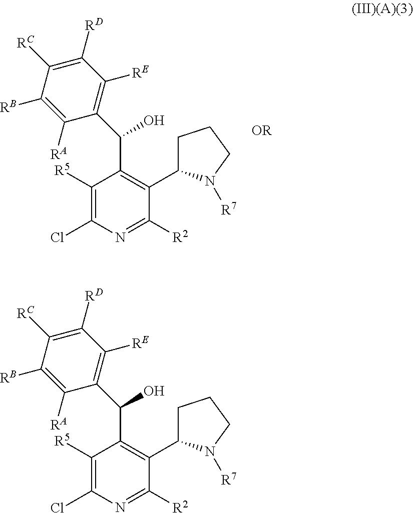

wherein:
R⁷ is H or alkyl;
R² and R⁵ are each independently H, alkyl, halo, alkoxy, aryl or heteroaryl; and
R^{A}, R^{B}, R^{C}, R^{D} and R^{E} are each independently halo.

In some embodiments of Formula (III)(A)(3), R^{A}, R^{B}, R^{C}, R^{D} and R^{E} are each fluoro, shown in Formula (III)(A)(3)(a):

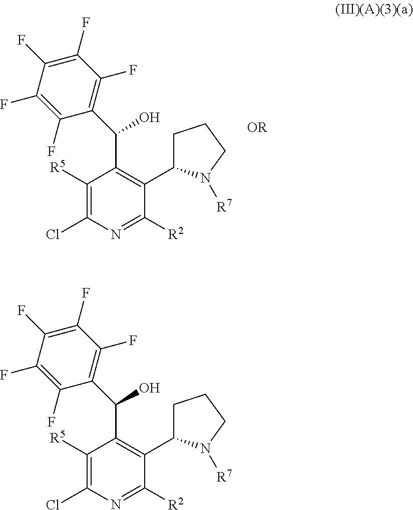

wherein:
R⁷ is H or alkyl; and
R² and R⁵ are each independently H, alkyl, halo, alkoxy, aryl or heteroaryl.

In some embodiments of Formula III, X is Cl; and R^{4a} and R^{4b} are each aryl; examples of which include, but are not limited to, Formula (III)(B)(1), Formula (III)(B)(2), Formula (III)(B)(3), and Formula (III)(B)(4):

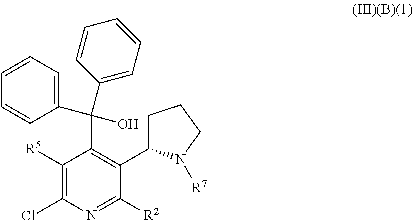

wherein:
R⁷ is H or alkyl; and
R² and R⁵ are each independently H, alkyl, halo, alkoxy, aryl or heteroaryl;

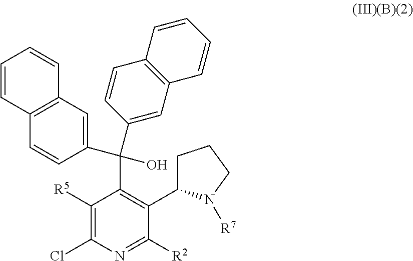

wherein:
R⁷ is H or alkyl; and
R² and R⁵ are each independently H, alkyl, halo, alkoxy, aryl or heteroaryl;

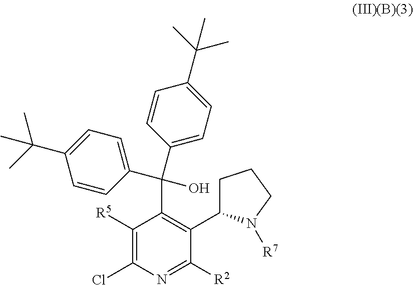

wherein:
R⁷ is H or alkyl; and
R² and R⁵ are each independently H. alkyl, halo, alkoxy, aryl or heteroaryl;

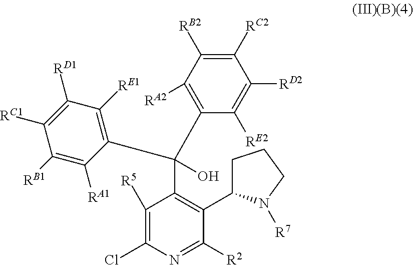

wherein:
R⁷ is H or alkyl;
R² and R³ are each independently H, alkyl, halo, alkoxy, aryl or heteroaryl; and
R^{A1}, R^{B1}, R^{C1}, R^{D1}, R^{E1}, R^{A2}, R^{B2}, R^{C2}, R^{D2} and R^{E2} are each independently halo.

In some embodiments, R^{A1}, R^{B1}, R^{C1}, R^{D1}, R^{E1}, R^{A2}, R^{B2}, R^{C2}, R^{D2} and R^{E2} are each fluoro, shown in Formula (III)(B)(4)(a):

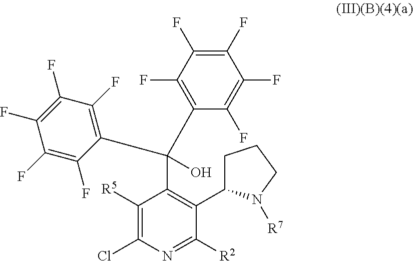

wherein:
R⁷ is H or alkyl; and
R² and R⁵ are each independently H, alkyl, halo, alkoxy, aryl or heteroaryl.

Also provided are amino alcohol compounds of Formula IV:

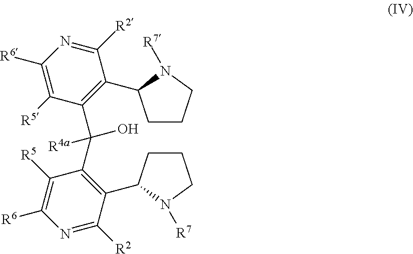

wherein:
R⁷ is H or alkyl;
R², R⁵ and R⁶ are each independently H, alkyl, halo, alkoxy, aryl or heteroaryl R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

Further provided are complexes comprising one or more of the amino alcohol compounds described above. As an example, the following is a zinc complex including an amino alcohol compound of Formula (III), wherein R² and R⁵ are each H, R⁷ is methyl, and X is Cl:

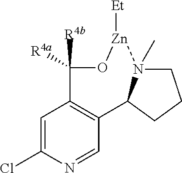

### 2. Phosphine Compounds

Provided herein are phosphine compounds of Formula X:

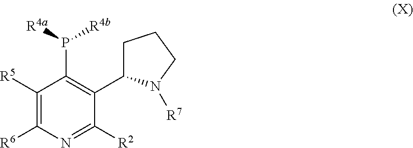

wherein:
R⁷ is H or alkyl;
R², R⁵ and R⁶ are each independently H, alkyl, halo, alkoxy, aryl or heteroaryl; and
R^{4a} and R^{4b} are each independently H, alkyl, aryl or heteroaryl.

Also provided herein are phosphine compounds of Formula XI:

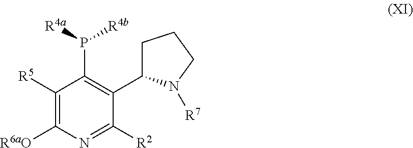

wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H. alkyl, aryl or heteroaryl.

Further provided herein are phosphine compounds of Formula XII:

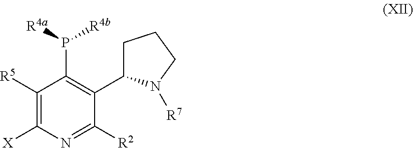

wherein:
R⁷ is H or alkyl;
R² and R⁵ are each independently H, alkyl, halo, alkoxy, aryl or heteroaryl;
X is halo; and
R^{4a} and R^{4b} are each independently H, alkyl, aryl or heteroaryl.

In some embodiments of Formula XII, X is Cl; and R^{4a} and R^{4b} are each aryl; examples of which include, but are not limited to, Formula (XII)(A):

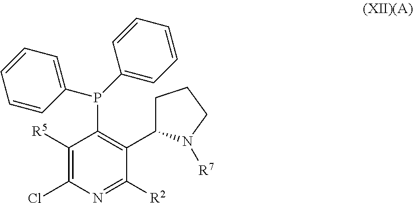

wherein:
R⁷ is H or alkyl; and
R² and R⁵ are each independently H, alkyl, halo, alkoxy, aryl or heteroaryl.

In some embodiments of Formula XII, X is Cl; and R^{4a} and R^{4b} are each alkyl; examples of which include, but are not limited to, Formula (XII)(B):

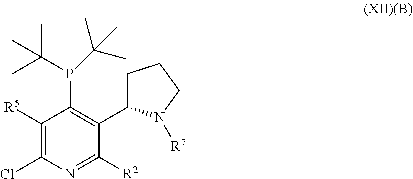

wherein:
R⁷ is H or alkyl; and
R² and R⁵ are each independently H, alkyl, halo, alkoxy, aryl or heteroaryl.

Further provided are phosphine compounds of Formula XIII:

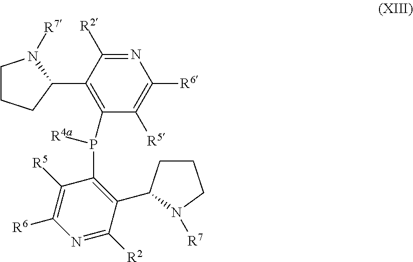

wherein:
R⁷ is H or alkyl;
R², R⁵ and R⁶ are each independently H, alkyl, halo, alkoxy, aryl or heteroaryl
R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

In some embodiments of Formula XIII, R⁶ and R^{6′} are each halo; R², R⁵, R^{2′} and R^{5′} are each H; and R^{4a} is aryl; an example of which includes, but is not limited to, Formula (XIII)(A):

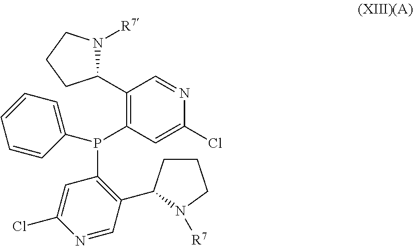

wherein:
R⁷ is H or alkyl; and

R^{7′} is H or alkyl.

In some embodiments of Formula XIII, R⁶ and R^{6′} are each halo; R², R⁵, R^{2′} and R^{5′} are each H; and R^{4a} is alkyl; an example of which includes, but is not limited to, Formula (XIII)(B):

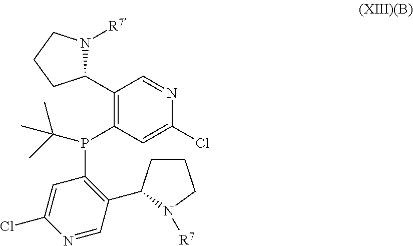

wherein:
R⁷ is H or alkyl; and

R^{7′} is H or alkyl.

Further provided are complexes comprising one or more of the phosphine compounds described above. As an example, the following is a complex with CuI that includes two phosphine compounds of Formula (XII)(A), wherein R⁷ and R^{7′} are each methyl:

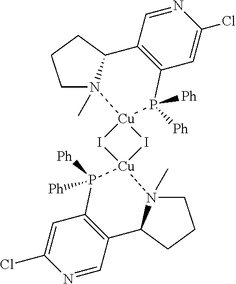

Some aspects of the present invention are described in more detail in the following non-limiting examples.

### EXAMPLES

### Example 1

### Synthesis of C-4 (S)—Nicotine-Based Amino Alcohol Derivatives

An alcohol function was installed at the C-4 position of (S)-6-chloronicotine to create sites for coordination to a metal. Compounds of this type were synthesized from commercially available (S)-nicotine in either two or three steps. Once synthesized, the (S)-6-chloronicotine-based derivatives were tested as catalysts in the well-known catalytic asymmetric addition of diethylzinc to benzaldehyde.

Compounds were synthesized from (5)-6-chloronicotine (63) (Scheme 1) in one step using the conditions shown in Table 1 to generate the lithiated C-4 species 72 from (S)-6-chloronicotine (63) (see U.S. Patent Publication No. 2007/0232665; Wagner, F. F.; Comins, D. L. Eur. J. Org. Chem. 2006, 3562-3565; Wagner, F. F.; Comins, D. L. Tetrahedron 2007, 63, 8065-8082). This direct method for substitution at the C-4 position of 63 was used for the synthesis of the bidentate ligands (Table 1).

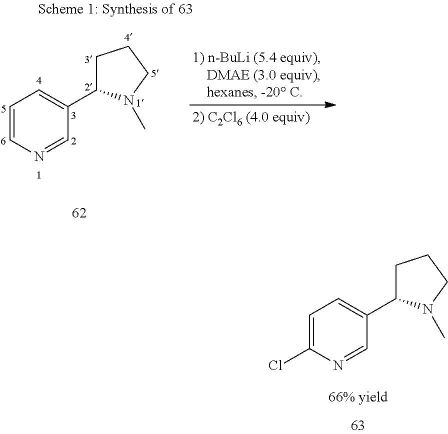

Selected aromatic aldehydes and ketones were used as electrophiles to produce secondary and tertiary alcohols, respectively, at the C-4 position of compound 63. Electrophiles containing aromatic rings were chosen to add steric bulk on the molecule close to the metal's binding site to help increase the enantioselectivity during the asymmetric reaction. Secondary and tertiary alcohols were compared to determine whether or not the enantioselectivity in the asymmetric reaction is affected by sterics and/or electronics resulting from the aromatic ring systems. The diastereomers (ratio of 1:1) at C-4 were separated by chromatography on silica gel to afford each enantiomerically pure diastereomer in low to moderate yields (Table 1).

TABLE 1
Formation of secondary and tertiary alcohols at the C-4 position of 63^{a}

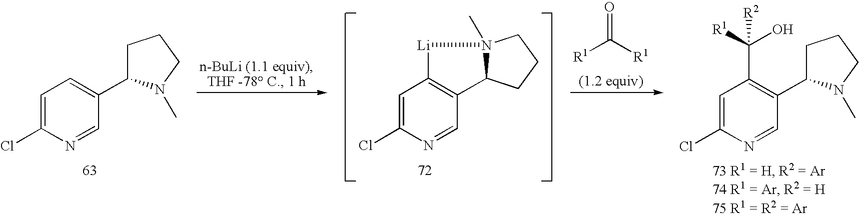

| entry | R¹COR² | yield 73 (%) | yield 74 (%) | yield 75 (%) |
|---|---|---|---|---|
| 1 | benzaldehyde | (S) 73a^{b}, 31 | (R) 74a^{b}, 37 | — |
| 2 | 2-naphthaldehyde | (S) 73b, 21 | (R) 74b, 25 | — |
| 3 | 1-naphthylaldehyde | (S) 73c, 12 | (R) 74c, 14 | — |
| 4 | pentafluorobenzaldehyde | (R) 73d, 25 | (S) 74d, 35 | — |
| 5 | benzophenone | — | — | 75a, 68 |
| 6 | di-naphthalen-2-yl methanone | — | — | 75b, 50 |
| 7 | bis-(4-tert-butylenyl)methanone | — | — | 75c, 39 |
| 8 | decafluorobenzophenone | — | — | 75d, 51 |

^{a}The absolute stereochemistry at the C-4 position of 73-74 was determined by the X-ray diffraction of 73a.
^{b}Known compound.³⁴

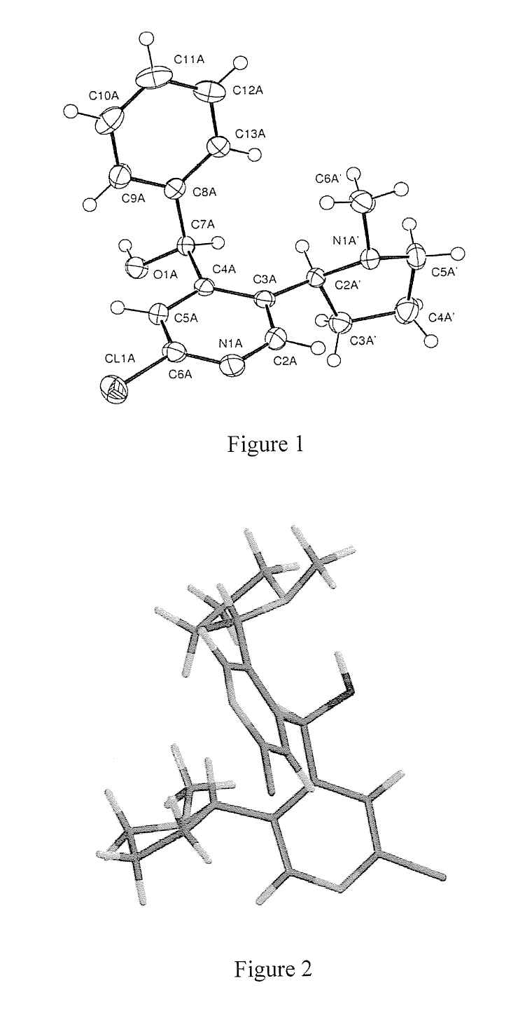

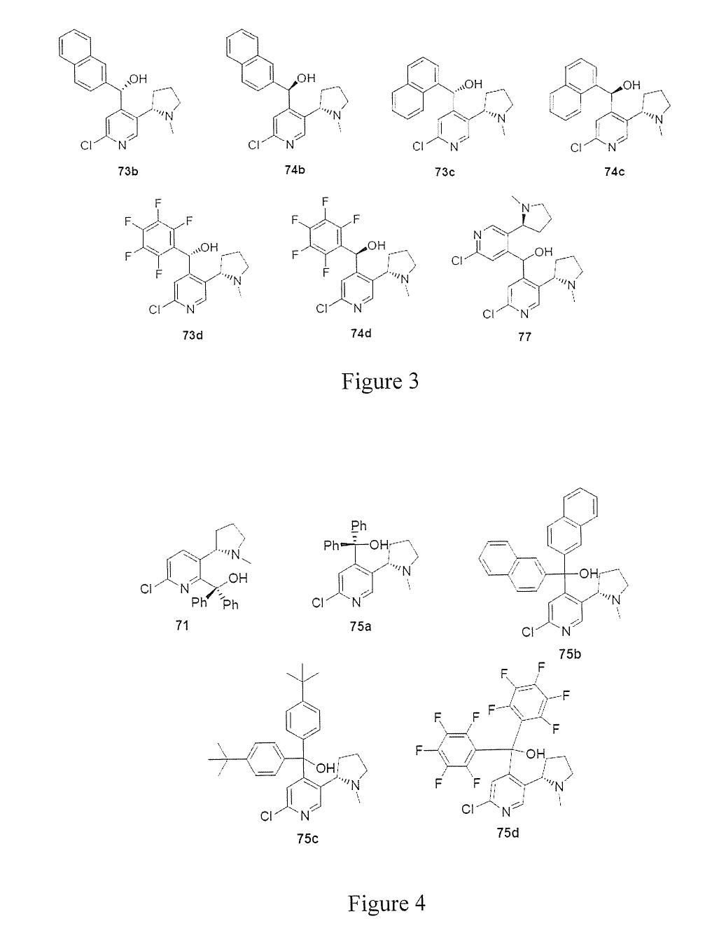

As shown in FIG. 1, the X-ray crystal structure defines the absolute stereochemistry at the C-4 position for diastereomer 73a. This assignment was applied to the other diastereomers for stereochemical determination as well. In addition to the aldehydes listed, triphenylacetaldehyde was also considered as a possible electrophile for the synthesis of another secondary alcohol; however, attempts to synthesize this aldehyde were unsuccessful.

Once again, using substitution at the C-4 position of 63, secondary alcohol derivative 77 was synthesized without the formation of diastereomers in 74% yield (Scheme 2). The aldehyde electrophile 76 was synthesized using ethyl formate for the formylation at C-4 (see U.S. Patent Publication No. 2007/0232665). During the characterization of 77, a doubling of peaks was seen in the ₁H and ₁₃C NMR spectra. To explain the peak doubling, 77 was modeled using the software Spartan (Johnson, J. A.; Deppmeier, B. J.; Driessen, A. J.; Hehre, W. J.; Klunzinger, P. E.; Pham, I. N.; Watanabe, M. Spartan '02 v 1.0.8. Irvine, Calif., 2002). The resulting model of the lowest energy confomer, depicted in FIG. 2, shows that the hydroxyl group can form a hydrogen bond with either pyrrolidine nitrogen causing the two identical substituents to become magnetically inequivalent, giving rise to the peak doubling.

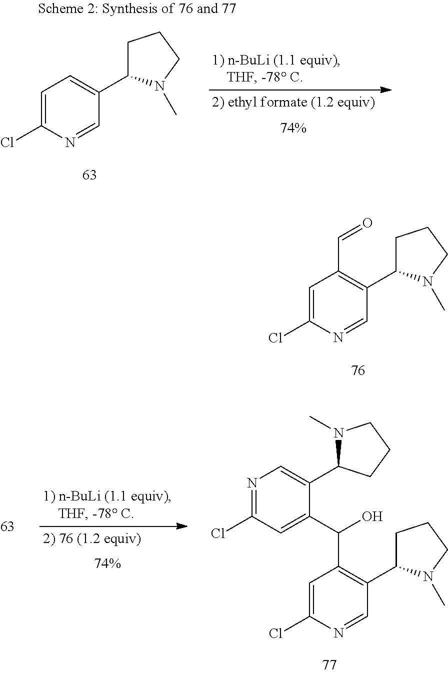

For synthesis of the tertiary alcohol derivatives, symmetrical ketones were used as electrophiles to avoid forming diastereomers that are difficult to separate and to increase the yield of the catalysts. This class of catalysts consisting of tertiary alcohols 75a-d was synthesized in low to moderate yields (Table 1).

Other symmetrical ketones, such as 9-fluorenone, di-napthylene-1-yl methanone and bis-(2-methoxyphenyl)methanone proved to be poor electrophiles for this reaction, and formation of their corresponding alcohols was unsuccessful. Insolubility of bis-biphenyl-4-yl-methanone in THF led to the variation of the solvent for the litihiation step. A D₂O quench was used to determine whether or not lithiation took place (Table 2). However, because the nonpolar ketone was also insoluble in DME, the corresponding amino alcohol was not synthesized.

TABLE 2
Variation of the solvent for C-4 lithiation

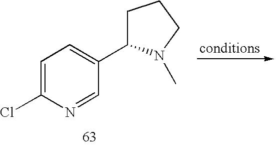

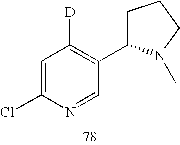

| entry | conditions | results |
|---|---|---|
| 1 | 1) n-BuLi (1.0 equiv). THF. −78° C. 1 h | crude ¹H NMR |
| | 2) D₂O | shows 78 |
| 2 | 1) n-BuLi (1.0 equiv). DME. −78° C. 1 h | crude ¹H NMR |
| | 2) D₂O | shows 78 |
| 3 | 1) n-BuLi (1.0 equiv) toluene. −42° C. 1 h | only 63 recovered |
| | 2) D₂O | |

All reactions were performed in flame-dried glassware under an argon atmosphere and stirred magnetically. Tetrahydrofuran (THF), diethyl ether, and toluene were distilled from sodium/benzophenone ketyl prior to use. Triethylamine, diisopropylamine, benzene, DMAE, DMF, and TMP were distilled from calcium hydride and stored under argon. Acetonitrile, methylene chloride, benzene, and hexanes were stored under argon over 4 Å molecular sieves. n-Butyllithium was titrated against diphenylacetic acid. Other reagents and solvents from commercial sources were stored under argon and used directly. Melting points were obtained from a Thomas-Hoover capillary melting point apparatus and are uncorrected. Radial preparative layer chromatography (RPLC) was performed on a Chromatotron (Harrison Associates, Palo Alto, Calif.) using glass plates coated with 1-, 2-, or 4-mm layers of Kieselgel 60 PF254 containing gypsum. High-resolution mass spectral analysis was performed at North Carolina State University. NMR spectra were obtained using a Varian Gemini GN-300 (300 MHz), Varian Mercury 300 (300 MHz), or Varian Mercury 400 (400 MHz) spectrometer. Chemical shifts are in δ units (ppm) with TMS (0.0 ppm) used as the internal standard for 1H NMR spectra and the CDCl3 absorption (77.2 ppm) for 13C NMR spectra. Chemical shifts for 19F NMR are reported from CFCl3 (0.0 ppm) where C6F6 (−163.0 ppm) was used as an external standard. All chemical shifts for 31P NMR are reported from H3PO4 (0.0 ppm). IR spectra were recorded on a Perkin-Elmer 1430 or Mattson Genesis II FTIR spectrometer. HPLC was performed using Waters and Associates (Milifrod, Mass.) 600 E multi solvent delivery system with a 486 tunable detector or a photodiode array detector equipped with a Chiralcel OD or a Chiralpak AD chiral analytical column. X-ray structure analysis was performed at North Carolina State University.

General procedure for the formation of the C-4 substituted (S)-6-chloronicotine alcohol derivatives. To a solution of (S)-6-chloronicotine (63, 200 mg, 1.02 mmol) in THF (2 mL) was added n-BuLi (0.68 mL, 1.02 mmol) at −78° C. After 1 h, a solution of the aldehyde (1.2 equiv) in THF (2 mL) kept over molecular sieves was cannulated into the reaction. The mixture was stirred for 30-60 min at −78° C. after which it was quenched with aqueous saturated sodium bicarbonate (2 mL). After warming to room temperature, the organic layer was separated. The aqueous layer was extracted with methylene chloride (2×10 mL). The combined organic layers were dried over potassium carbonate, filtered, and concentrated in vacuo. Yields are shown in Table 1.

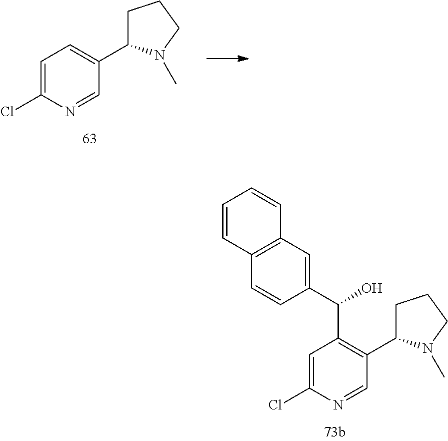

(1S)[5-((2S)-1-Methylpyrrolidin-2-yl)-2-chloro(4-pyridyl)]-2-naphthylmethan-1-ol76 (73b). Purified by RPLC (1% TEA/20% EtOAc/hexanes) to give a white solid, mp 60-62° C.; [α]31D −19.8 (c 2.15, CH2Cl2); IR (thin film) 3287, 3057, 2966, 2843, 2789, 1585, 1458, 1372, 1092, 1042 cm−1; 1H NMR (400 MHz, CDCl3) δ 8.36 (s, 1H), 8.25 (s, 1H), 7.96 (s, 1H), 7.90-7.86 (m, 3H), 7.54-7.50 (m, 2H), 7.39-7.36 (m, 1H), 6.66 (s, 1H), 6.26 (s, 1H), 3.45-3.40 (m, 1H), 3.37-3.33 (m, 1H), 2.49-2.38 (m, 2H), 2.35-2.11 (m, 5H), 2.07-2.00 (m, 1H); 13C NMR (100 MHz. CDCl3) δ 155.9, 152.2, 151.2, 137.5, 134.2, 133.6, 133.3, 128.5, 128.4, 128.0, 126.5, 126.4, 126.1, 125.3, 124.2, 71.2, 69.7, 57.2, 40.6, 32.5, 24.3; HRMS calcd for C21H21ClN2O ([M+H]+) 353.1421, found 353.1436.

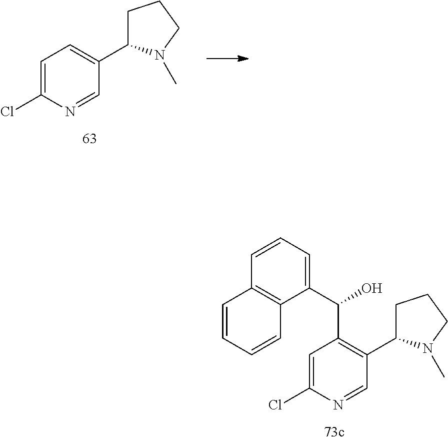

(1S)[5-((2S)-1-Methylpyrrolidin-2-yl)-2-chloro(4-pyridyl)]naphthylmethan-1-ol76 (73c). Purification by RPLC (1% TEA/20% EtOAc/hexanes) afforded a colorless oil; [α]31 D −19.8 (c 2.15, CH2Cl2); IR (neat) 3275, 3055, 2967, 2790, 1585, 1460, 1376, 1221, 1089, 790.7 cm-1; 1H NMR (400 MHz, CDCl3) δ 8.58 (s, 1H), 8.28 (s, 1H), 7.93-7.88 (m, 3H), 7.63-7.59 (m, 99 1H), 7.49-7.44 (m, 1H), 7.41-7.35 (m, 2H), 6.70 (s, 1H), 6.49 (s, 1H), 3.50-3.46 (m, 1H), 3.39-3.35 (m, 1H), 2.63-2.40 (m, 3H), 2.31 (s, 3H), 2.27-2.14 (m, 1H), 2.11-2.03 (m, 1H); 13C NMR (100 MHz, CDCl3) δ 155.61, 152.44, 151.35, 135.45, 133.96, 133.87, 130.58, 129.35, 128.82, 126.49, 125.97, 125.73, 125.23, 123.90, 123.49, 69.90, 68.04, 57.32, 40.70, 32.57, 24.69; HRMS calcd for C21H21ClN2O ([M+H]+) 353.1421, found 353.1429.

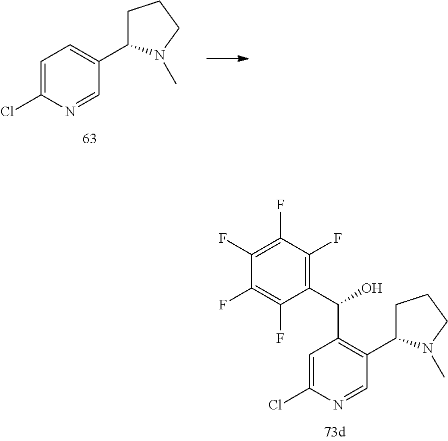

(1R)[5-((2S)-1-Methylpyrrolidin-2-yl)-2-chloro(4-pyridyl)](2,3,4,5,6-pentafluorophenyl)methan-1-ol77 (73d). The crude mixture was purified by RPLC (5% TEA/hexanes) and the diastereomers were separated by RPLC with CH2Cl2 to afford a white solid, mp 113-115° C.; [α]32 D −89.3 (c 2.45, CH2Cl2); IR (thin film) 3227, 2968, 2843, 2793, 1652, 1585, 1522, 1504, 1462, 1120, 996, 961 cm−1; 1H NMR (400 MHz, CDCl3) δ 9.51 (s, 1H), 8.28 (s, 1H), 6.76 (s, 1H), 6.42 (s, 1H), 3.44-3.40 (m, 1H), 3.34-3.301 (m, 1H), 2.46-2.34 (m, 2H), 2.29 (s, 3H), 2.23-2.12 (m, 2H), 2.10-2.02 (m, 1H); 13C NMR (75 MHz, CDCl3) δ 152.5, 152.4, 151.9, 147.0, 143.8, 143.2, 140.0, 136.5, 133.7, 122.6, 115.5, 70.1, 65.7, 56.9, 40.4, 32.4, 23.8; 19F NMR (282 MHz, CDCl3) δ-137.1 (m, 2F), −149.7 (m, 1F), −157.3 (m, 2F); HRMS calcd for C17H14ClF5N2O ([M+H]+) 393.0788, found 393.0791.

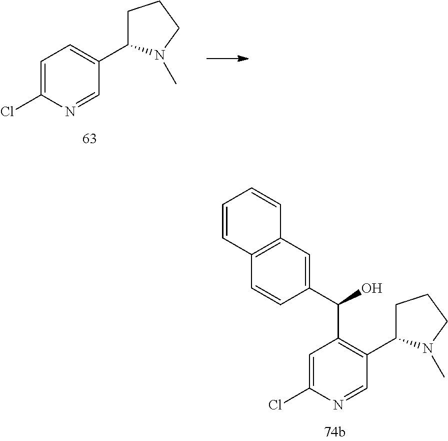

(1R)[5-((2S)-1-Methylpyrrolidin-2-yl)-2-chloro(4-pyridyl)]-2-naphthylmethan-1-ol76 (74b). Purified by RPLC (1% TEA/20% EtOAc/hexanes) to give a thick, pale yellow oil; [α]34 D+21.5 (c 1.20, CHCl3); IR (neat) 3273, 3057, 2966, 2839, 2786, 1585, 1458, 1372, 1150, 1092, 1041 cm−1; 1H NMR (400 MHz, CDCl3) δ 8.34 (s, 1H), 7.83-7.77 (m, 4H), 7.53-7.46 (m, 4H), 7.34-7.32 (m, 1H), 6.02 (s, 1H), 3.28-3.19 (m, 2H), 2.27-2.20 (m, 4H), 1.66-1.51 (m, 3H), 1.25-1.13 (m, 1H); 13C NMR (100 MHz, CDCl3) δ 154.2, 151.6, 151.2, 140.7, 134.5, 133.3, 133.0, 128.6, 128.2, 127.9, 126.6, 126.4, 125.3, 124.6, 124.5, 75.1, 68.7, 56.5, 40.8, 32.7, 22.4; HRMS calcd for C21H21ClN2O ([M+H]+) 353.1421, found 353.1439.

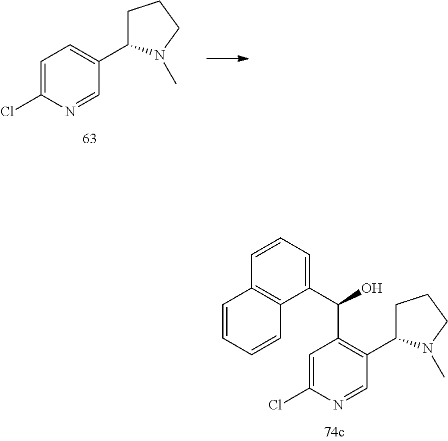

(1R)[5-((2S)-1-Methylpyrrolidin-2-yl)-2-chloro(4-pyridyl)]naphthylmethan-1-ol76 (74c). Purification by RPLC (1% TEA/20% EtOAc/hexanes) afforded the product as a foam; [α]31 D −115.2 (c 1.80, CH2Cl2); IR (neat) 3273, 3057, 2966, 2839, 2786, 1586, 1459, 1374, 1092, 786 cm−1; 1H NMR (400 MHz, CDCl3) δ 8.43 (s, 1H), 8.11-8.08 (m, 1H), 7.91-7.89 (m, 1H), 7.84-7.82 (d, J=8.0 Hz, 1H), 7.54-7.49 (m, 2H), 7.41-7.37 (m, 1H), 7.27 (s, 1H), 7.17-7.16 (m, 1H), 6.71 (s, 1H), 5.45 (s, 1H), 3.32-3.28 (m, 1H), 3.11-3.07 (m, 1H), 2.23-2.16 (m, 4H), 1.87-1.69 (m, 2H), 1.64-1.54 (m, 2H); 13C NMR (100 MHz, CDCl3) δ 154.1, 150.8, 150.5, 137.5, 134.9, 134.3, 131.0, 129.4, 129.2, 126.9, 126.3, 126.1, 125.4, 123.8, 122.6, 70.0, 66.7, 56.3, 40.7, 33.4, 22.5; HRMS calcd for C21H21ClN2O ([M+H]+) 353.1421, found 353.1447.

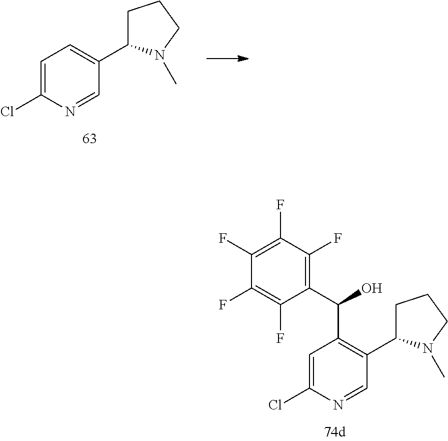

(1S)[5-((2S)-1-Methylpyrrolidin-2-yl)-2-chloro(4-pyridyl)](2,3,4,5,6-pentafluorophenyl)methan-1-ol77 (74d). The crude mixture was purified by RPLC (5% TEA/hexanes) and the diastereomers were separated by RPLC with CH2Cl2 to afford a white solid, mp 136-138° C.; [α]32 D −109.8 (c 2.65, CH2Cl2); IR (thin film) 3213, 2970, 2843, 2791, 1653, 1587, 1522, 1504, 1460, 1119, 996, 962 cm−1; 1H NMR (400 MHz, CDCl3) δ 9.05 (s, 1H), 8.32 (s, 1H), 6.90 (s, 1H), 6.32 (s, 1H), 3.42-3.38 (m, 1H), 3.29-3.25 (m, 1H), 2.43-2.36 (m, 1H), 2.30 (s, 3H), 2.25-2.16 (m, 1H), 2.16-2.07 (m, 1H), 2.04-1.95 (m, 1H), 1.92-1.83 (m, 1H); 13C NMR (75 MHz, CDCl3) δ 152.5, 152.1, 151.4, 147.0, 143.7, 140.0, 139.7, 136.4, 133.3, 122.2, 117.0, 69.7, 66.1, 56.5, 40.3, 34.0, 22.2; 19F NMR (376 MHz, CDCl3) δ 6-137.9 (m, 2F), −149.8 (m, 1F), −157.4 (m, 2F); HRMS calcd for C17H14ClF5N2O ([M+H]+) 393.0788, found 393.0790.

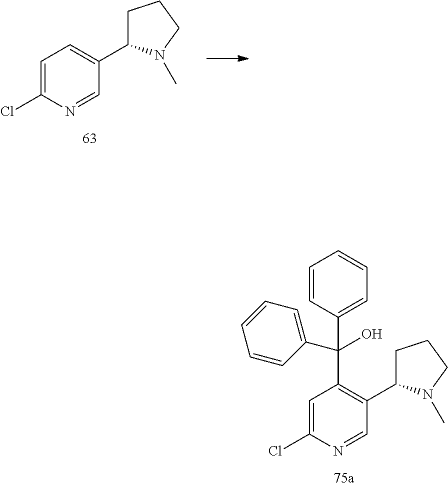

[5-((2S)-1-Methylpyrrolidin-2-yl)-2-chloro(4-pyridyl)]diphenylmethan-1-ol (75a). The product was purified by RPLC (1% TEA/20% ethyl acetate/hexanes) to afford a white solid, nip 132-134° C.; [α]30 D −86.0 (c 1.70, CHCl3); IR (thin layer) 3301, 3087, 3060, 3029, 2961, 2877, 2841, 2779, 1575, 1449, 1373, 1325, 1286, 1148, 1043, 905.2, 763.5 cm−1; 1H NMR (400 MHz, CDCl3) δ 10.20 (s, 1H), 8.28 (s, 1H), 7.32-7.16 (m, 10H), 6.72 (s, 1H), 3.37-3.38 (m, 1H), 3.19-3.16 (m, 1H), 2.31-2.24 (m, 1H), 2.05-1.71 (m, 4H), 1.81 (s, 3H); 13C NMR (100 MHz, CDCl3) δ 158.3, 152.6, 150.7, 147.4, 147.1, 133.9, 128.2, 128.1, 128.0, 127.7, 127.6, 127.0, 126.7, 83.0, 71.2, 56.4, 40.1, 33.6, 22.2; HRMS calcd for C23H23ClN2O ([M+H]+) 379.1577, found 379.1579.

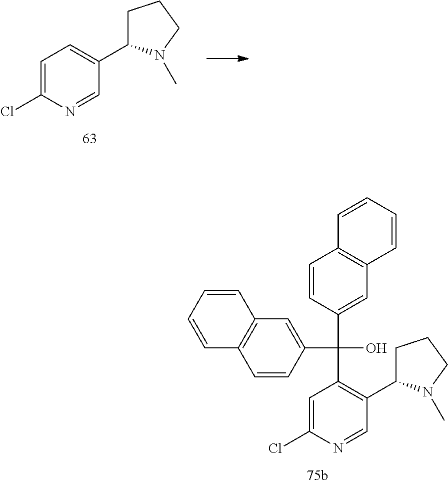

[5-((2S)-1-Methylpyrrolidin-2-yl)-2-chloro(4-Pyridyl)]di-naphthalen-2-ylmethan-1-ol (75b). The product was purified by RPLC (1% TEA/10% ethyl acetate/hexanes) to afford a white solid, mp 236-238° C.; [α]32 D −125.0 (c 1.15, CH2Cl2); IR (thin layer) 3294, 3057, 2957, 2786, 1575, 1505, 1461, 1146, 1121, 907 cm−1; 1H NMR (400 MHz, CDCl3) δ 10.67 (s, 1H), 8.34 (s, 1H), 7.87-7.82 (m, 4H), 7.71-7.70 (m, 2H), 7.61-7.59 (m, 1H), 7.53-7.43 (m, 7H), 6.87 (s, 1H), 3.42-3.38 (m, 1H), 3.23-3.19 (m, 1H), 2.32-2.26 (m, 1H), 2.07-1.80 (m, 6H), 1.77-1.72 (m, 1H); 13C NMR (100 MHz, CDCl3) δ 157.86, 152.83, 151.00, 144.75, 144.64, 134.06, 132.92, 132.86, 132.81, 132.78, 128.67, 128.65, 128.29, 128.26, 127.74, 127.71, 127.14, 126.99, 126.94, 126.57, 126.52, 126.47, 126.45, 126.38, 126.36, 83.40, 71.29, 56.42, 40.23, 33.53, 22.21; HRMS calcd for C31H21ClN2O ([M+H]+) 479.1890, found 479.1919.

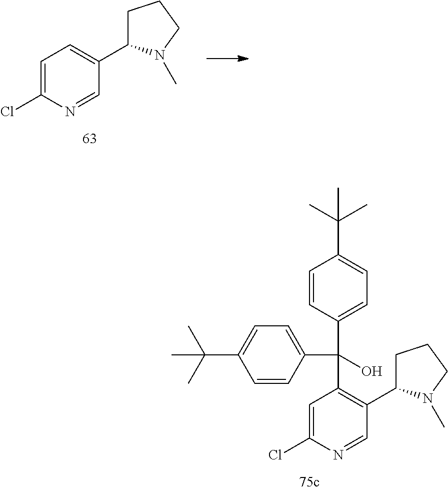

[5-((2S)-1-Methylpyrrolidin-2-yl)-2-chloro(4-pyridyl)]bis-(4-tert-butylphenyl)methan-1-ol (75c). The product was purified by RPLC (1% TEA/1% MeOH/CH2Cl2) followed by recrystallization in hexanes to afford a white solid, mp 226-230° C.; [α]33 D −98.5 (c 1.50, CH2Cl2); IR (thin film) 3342, 3036, 2961, 2871, 2784, 1575, 1461, 1369, 1149, 1110, 733 cm−1; 1H NMR (400 MHz, CDCl3) δ 9.78 (s, 1H), 8.27 (s, 1H), 7.32-7.25 (m, 5H), 7.15-7.05 (m, 3H), 6.80 (s, 1H), 3.35-3.31 (m, 1H), 3.19-3.15 (m, 1H), 2.29-2.22 (m, 1H), 2.06-1.88 (m, 3H), 1.78-1.70 (m, 4H), 1.29 (s, 18H); 13C NMR (100 MHz, CDCl3) δ 158.62, 152.57, 150.53, 150.45, 150.41, 144.48, 143.97, 134.17, 127.77, 127.71, 126.88, 125.08, 125.03, 82.78, 71.28, 56.54, 40.05, 34.64, 34.00, 31.53, 22.21; HRMS calcd for C31H39ClN2O (M+) 491.2829, found 491.2845.

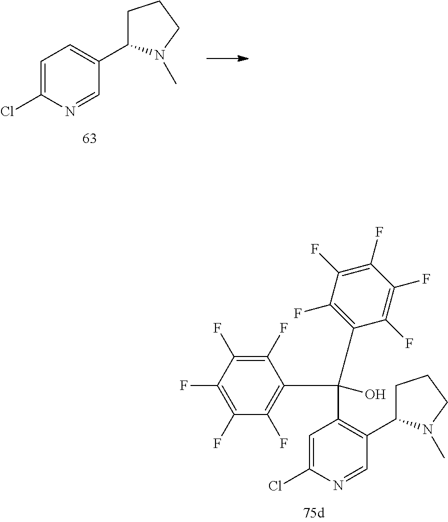

[5-((2S)-1-Methylpyrrolidin-2-yl)-2-chloro(4-pyridyl)]bis(2,3,4,5,6-pentafluorophenyl)methan-1-ol76,77 (75d). The crude mixture was purified using RPLC (5% TEA/hexanes and CH2Cl2) to afford 146 mg (51%) of 75d as a white solid, mp 38-40° C.; [α]32 D −16.9 (c 1.10, CH2Cl2); IR (thin film) 3380, 2963, 2924, 2856, 1650, 1578, 1524, 1484, 1122, 1005 cm−1; 1H NMR (400 MHz, CDCl3) δ 12.82 (s, 1H), 8.36 (s, 1H), 6.93 (s, 1H), 3.54-3.49 (m, 1H), 3.35-3.30 (m, 1H), 2.49-2.41 (m, 1H), 2.27 (s, 3H), 2.24-2.17 (m, 1H), 2.13-1.96 (m, 2H), 1.93-1.85 (m, 1H); 13C NMR (75 MHz, CDCl3) δ 154.3, 153.2, 152.5, 146.7, 143.4, 142.9, 139.8, 139.7, 136.7, 136.5, 132.6, 125.3, 79.5, 71.4, 56.3, 39.7, 32.7, 22.4; 19F NMR (282 MHz, CDCl3) δ−133.2 (d, J=18.6 Hz, 2F), −133.9 (d, J=18.6 Hz, 2F), −149.7 (m, 2F), −157.4 (m, 4F); HRMS calcd for C23H13ClF₁₀N2O ([M+H]+) 559.0635, found 559.0654.

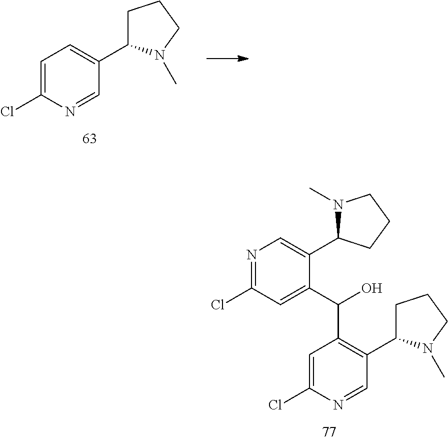

Bis[5-((2S)-1-methylpyrrolidin-2-yl)-2-chloro-4-pyridyl]methan-1-ol (77). To a solution of n-BuLi (2.35 M in hexanes, 0.34 mL) in THF (2 mL) was added (S)-6-chloronicotine (63, 158 mg, 0.80 mmol) at −78° C. After 1 h, a solution of (S)-6-chloro-4-formylnicotine (217 mg, 0.96 mmol) in THF (2 mL) was added dropwise into the reaction. The mixture was slowly warmed from −78° C. to −30° C. over 1 h after which aqueous saturated sodium bicarbonate (2 mL) was added. The aqueous layer was extracted with methylene chloride (3×10 mL). The combined organic layers were dried over potassium carbonate, filtered, and concentrated. The crude mixture was purified using RPLC (1% TEA/30% ethyl acetate/hexanes) to afford 249 mg (74%) of 77 as a white solid, mp 80-82° C.; [α]31D −100.6 (c 0.95, CH2Cl2); IR (thin film) 3238, 2967, 2876, 2840, 2787, 1582, 1455, 1372, 1146, 1092 cm−1; 1H NMR (400 MHz, CDCl3) δ 8.92 (s, 1H), 8.54 (s, 1H), 8.24 (s, 1H), 7.67 (s, 1H), 6.41 (s, 1H), 6.31 (s, 1H), 3.46-3.42 (m, 1H), 3.38-3.32 (m, 1H), 3.10-3.06 (m, 1H), 2.89-2.85 (m, 1H), 2.51-2.41 (m, 2H), 2.33-2.20 (m, 4H), 2.16-2.03 (m, 3H), 1.98 (s, 3H), 1.92-1.79 (m, 1H), 1.69-1.56 (m, 3H); 13C NMR (75 MHz, CDCl3) d 154.1, 152.4, 151.6, 151.3, 150.7, 135.1, 133.6, 123.6, 122.7, 69.8, 67.0, 66.3, 57.3, 57.1, 40.7, 40.6, 34.5, 31.9, 24.6, 22.9; HRMS calcd for C21H26Cl2N₄₀ (M+) 421.1556, found 421.1554.

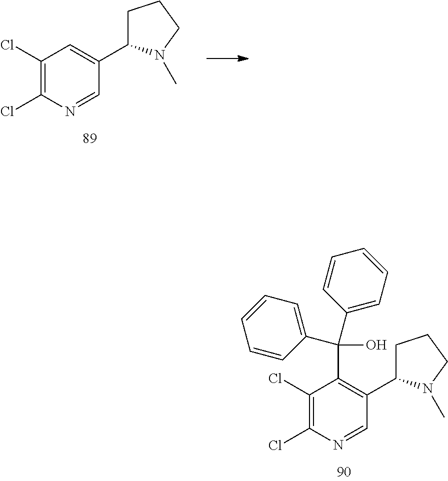

[5-((2S)-1-Methylpyrrolidin-2-yl)-2,3-dichloro(4-pyridyl)]diphenylmethan-1-ol (90). To a solution of n-BuLi (0.18 mL, 0.40 mmol) in THF (1 mL) was added (S)-5,6-dichloronicotine28 (89, 85 mg, 0.37 mmol) at −78° C. After 1 h, a solution of benzophenone (87 mg, 0.48 mmol) in THF (1 mL) kept over molecular sieves was added dropwise into the reaction at −78° C. After warming to −20° C., the reaction was stirred for 1 h and quenched with aqueous saturated sodium bicarbonate (2 mL). The aqueous layer was extracted with methylene chloride (2×10 mL). The combined organic layers were dried over potassium carbonate, filtered, and concentrated. The crude mixture was purified using RPLC (1% TEA/2% EtOAc/hexanes and CH2Cl2) to afford 46 mg (30%) of 90 as a white solid, mp 170-172° C.; [α]32 D −164.54 (c 1.10, CH2Cl2); IR (thin film) 3321, 3057, 3027, 2957, 2784, 1550, 1489, 1447, 1310, 1219, 1057 cm−1; 1H NMR (400 MHz, CDCl3) δ 10.19 (s, 1H), 8.30 (s, 1H), 7.41-7.31 (m, 5H), 7.26-7.21 (m, 3H), 7.12-7.10 (m, 2H), 3.48-3.43 (m, 1H), 3.12-3.08 (m, 1H), 2.64-2.55 (m, 1H), 2.34-2.28 (m, 1H), 2.26-2.19 (m, 1H), 2.12-2.00 (m, 1H), 1.89-1.83 (m, 1H), 1.64 (s, 3H); 13C NMR (100 MHz, CDCl3) δ 157.4, 150.4, 147.4, 143.2, 136.7, 129.0, 128.2, 128.0, 127.9, 127.8, 127.1, 84.8, 73.2, 56.5, 39.4, 34.8, 22.0; HRMS calcd for C23H22Cl2N2O (M+) 413.1181, found

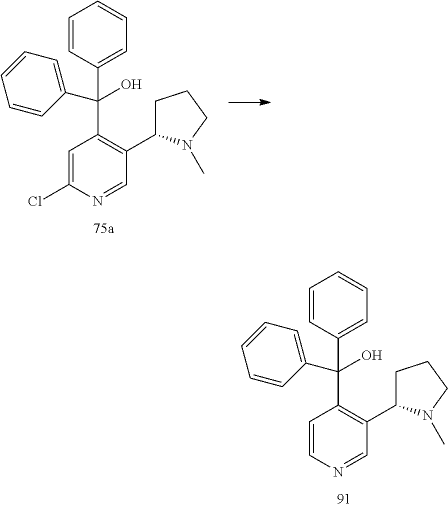

[3-((2S)-1-Methylpyrrolidin-2-yl)(4-pyridyl)]diphenylmethan-1-ol (91). After adding methanol (5 mL) to a flask containing [5-((2S)-1-methylpyrrolidin-2-yl)-2-chloro(4-pyridyl)]diphenylmethan-1-ol (75a, 50 mg, 0.132 mmol, 1.0 equiv), Pearlman's catalyst (20 wt. % Pd on carbon, 18 mg, 0.026 mmol, 0.20 equiv), sodium hydroxide (21 mg, 0.528 mmol, 4.0 equiv), hydrogen was backfilled into the flask following evacuation under vacuum. The reaction was stirred at room temperature under balloon pressure of hydrogen for 1 h. The mixture was then filtered and concentrated, and the remaining solid was then dissolved in ethyl acetate. After drying over potassium carbonate, the organic layer was filtered and concentrated. The crude mixture was purified by RPLC (1% TEA/50% EtOAc/hexanes) to afford 45 mg (99%) of 91 as a white solid, mp 232-234° C.; [α]31D −113.4 (c 0.95, CH2Cl2); IR (thin film) 3125, 2959, 2933, 2824, 2774, 1593, 1444, 1197, 1042, 764 cm−1; 1H NMR (400 MHz, CDCl3) δ 10.43 (s, 1H), 8.50 (s, 1H), 8.34-8.32 (d, J=8 Hz, 1H), 7.30-7.22 (m, 8H), 7.19-7.17 (m, 2H), 6.71-6.69 (d, J=8 Hz, 1H), 3.39-3.34 (m, 1H), 3.22-3.18 (m, 1H), 2.32-2.25 (m, 1H), 2.10-1.86 (m, 3H), 1.83 (s, 3H), 1.80-1.72 (m, 1H); 13C NMR (100 MHz, CDCl3) δ 154.7, 153.1, 148.3, 148.2, 147.8, 134.6, 128.2, 128.0, 127.4, 127.3, 127.0, 83.1, 72.1, 56.5, 40.1, 33.7, 22.2; HRMS calcd for C23H24N2O ([M+H]+) 345.1961, found 345.1965.

### Example 2

### Testing Amino-Alcohol Catalysts in the Catalytic Asymmetric Addition of Diethylzinc to Aldehydes

The ligands that were synthesized and characterized were screened for use as catalysts in the asymmetric addition of diethylzinc to benzaldehyde. All reactions were run in toluene and kept at 0° C. for 24 h (Table 3).

General procedure for the catalytic asymmetric addition of diethylzinc to benzaldehyde. To a stirred solution of the catalyst (0.20 equiv) in toluene (2 mL) at 0.0 was added diethylzinc (1.0 M in hexane, 1.08 mL). After stirring the reaction for 30 min at 0° C., benzaldehyde (0.05 mL, 0.49 mmol) was added and the reaction was stirred at this temperature for 24 h. The reaction was quenched with an aqueous saturated solution of ammonium chloride (2 mL), and the mixture was extracted with diethyl ether (3×5 mL). The combined organic layers were dried over MgSO4, filtered, and concentrated to afford the crude product. Purification by RPLC (10% EtOAc/hexanes) provided the product as a colorless oil. The ee of the alcohol products was determined by chiral HPLC.

(R)-1-Phenyl-1-propanol (17): [α]31D +37.8 (c 1.70, CH2Cl2). 95% ee by HPLC analysis (Chiralcel OD column, 2% i-propanol/hexanes, 1.00 mL/min, λ=219 nm). Retention times: R (major) 11.98 min, S (minor) 14.95 min.

(R)-1-(4-Chlorophenyl)-1-propanol: [α]31D +31.5 (c 1.90, CH2Cl2). 94% ee by HPLC analysis (Chiralcel OD column, 2% i-propanol/hexanes, 0.80 mL/min. λ=254 nm). Retention times: R (major) 15.04 min, S (minor) 16.41 min.

(R)-1-(4-Methoxyphenyl)-1-propanol: [α]31D+28.3 (c 0.70, CH2Cl2). 94% ee by HPLC analysis (Chiralcel OD column, 2% i-propanol/hexanes. 1.00 mL/min, λ=261 nm). Retention times: R (major) 45.09 min. S (minor) 55.69 min.

(R)-1-Phenyl-3-pentanol: [α]31D −18.6 (c 1.25, CH2Cl2). 69% ee by HPLC analysis (Chiralcel OD column, 5% i-propanol/hexanes, 1.00 mL/min, =262 nm). Retention times: R (major) 9.14 min, S (minor) 12.94 min.

(R)-1-Phenylpent-1-(E)-en-3-ol: [α]31D −1.0 (c 2.60, CH2Cl2); 72% ee by HPLC analysis (Chiralcel OD column eluted with 5% i-propanol/hexanes at 0.80 mL/min and detection at λ=254 nm). Retention times: R (major) 14.97 min, S (minor) 24.68 min.

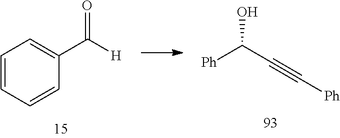

(S)-1,3-Diphenyl-prop-2-yn-1-ol (93). Diethylzinc (1.0 M in hexane, 0.39 mL) was added dropwise to a solution of 75d (11 mg, 0.02 mmol) in toluene (2 mL) at room temperature. After 15 h, phenylacetylene (0.06 mL, 0.41 mmol) was added at room temperature and the reaction was stirred for 1 h. Benzaldehyde (0.02 mL, 0.20 mmol) was then added and the reaction was stirred for 24 h at room temperature. The reaction was quenched with water and extracted with diethyl ether (2×5 mL). The combined organic layers were dried over magnesium sulfate, filtered, and concentrated. The crude oil was purified by RPLC (10% EtOAc/hexanes) to afford 93% (38 mg) of the product as a colorless oil; 13% ee determined by HPLC analysis (Chiralcel OD column eluted with 20% i-propanol/hexanes at 1.00 mL/min and detection at λ=254 nm). Retention times: R (minor) 7.50 min, S (major) 10.48 min. 40a

Toluene proved to be the better solvent for the reaction since a slight decrease in ee to 72% was observed when catalyst 75a was used in cyclohexane. Conditions where an additive, such as titanium(IV) isopropoxide, were also attempted with catalyst 75a, but both a low yield and low ee's were obtained (4% and 6%, respectively). Only results from the standard reaction conditions are shown in Table 14.

Both diastereomers of the secondary alcohols were examined as catalysts in the reaction, shown in entries 2-9. Interestingly, only diastereomers with the absolute configuration of 74a-d had a significant effect on the enantioselectivity. In addition, in every case where a significant enatioselectivity was observed, the primary product was the R enantiomer.

Compound 74d (entry 9) gave the highest enantiomeric excess for this class of ligands; however, the 1-naphthyl substituted ligand 74c provided a similar ee of 76% with an 83% yield. In the case of 74d. the electron-withdrawing effect of the fluorines on the aromatic ring created a decrease in the yield as compared to 74a-c.

When comparing the selectivity provided by catalysts 74c and 74d (76% ee and 79% ee, respectively) with 74a and 74b (both 64% ee) it appears that selectivity can be increased by either adding more electron withdrawing substituents or increasing the steric hindrance. These findings were also observed in the series of tertiary alcohols 75a-d. The decafluorobenzophenone tertiary alcohol 75d gave a 95% ee, while the benzophenone tertiary alcohol 75a only gave 79% ee (entries 10 and 13). The greater steric bulk of 75b gave an ee of 83%, while the smaller tertiary alcohol 75c only afforded 66% ee (entries 11 and 12).

TABLE 3
Secondary and tertiary alcohols as ligands in the catalytic
asymmetric addition of diethylzinc to benzaldehyde

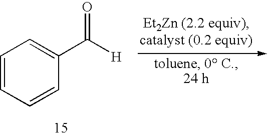

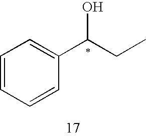

| entry | catalyst | yield (%) | ee^{a}(%) | |
|---|---|---|---|---|
| 1 | 71 | 42 | 2 | (R) |
| 2 | 73a | 82 | 14 | (R) |
| 3 | 74a | 64 | 64 | (R) |
| 4 | 73b | 13 | 2 | (S) |
| 5 | 74b | 73 | 64 | (R) |
| 6 | 73c | 90 | 10 | (S) |
| 7 | 74c | 83 | 76 | (R) |
| S | 73d | 64 | 7 | (R) |
| 9 | 74d | 48 | 79 | (R) |
| 10 | 75a | 78 | 79 | (R) |
| 11 | 75b | 83 | 8 | (R) |
| 12 | 75c | 72 | 66 | (R) |
| 13 | 75d | 66^{b} | 95 | (R) |
| 14 | 77 | 91 | 67 | (R) |

^{a}Detennined by chiral HPLC on column Chiralcel OD with λ = 219 nm. 10% i-propanol/hexanes as the eluent with a flow rate of 1.00 mL/min.
^{b}Lower yield due to difficulties in purification.

In general, the tertiary alcohols, entries 10, 11, and 13, enhanced the selectivity of the reaction more than the secondary alcohols. The decafluoro tertiary alcohol 75d proved to provide one of the best enantioselectivities (95% ee) overall, but gave the lowest yield.

Surprisingly, the increased steric bulk provided by the tert-butyl groups on compound 75c did not improve the selectivity over that afforded by 75d and even decreased the selectivity when compared to the smaller compound 75a. Gau and Wu discovered with their 13-amino alcohol systems that aromatic substituents increased the ee, while alkyl substituents decreased the ee (Gau, H. M.; Wu, K. H. Organometallics 2003, 22, 5193-5200). Their finding is consistent with the results in Table 14 when comparing the tertiary and secondary alcohols. For example, the additional phenyl group on 75a (Table 3, entry 10) causes an increase in the selectivity by about 10% ee over that of entry 3. This shows that increase in steric bulk of the catalyst does help to increase enantioselectivity. However, catalyst 75b did not seem to show much of an increase in yield or selectivity from 75a suggesting that the 2-naphthyl substituents do not increase the sterics much from the phenyl substituents.

Upon determining 75d as the catalyst with the highest selectivity at 20 mol %, the asymmetric reaction parameters were optimized by varying the conditions of the reaction by lowering the catalyst loading (Table 4). When going from 20 mol % to 10 mol % of catalyst 75d, only a slight change in percent yield and percent ee were observed. Much to our delight, the reaction went further to completion when 5 mol % of catalyst 75d was employed in the reaction than when 10% was used. However, when the amount of catalyst was further reduced to 2 mol % of 75d, a decrease in the yield and selectivity was observed (Table 4, entry 4).

TABLE 4
Variation of the catalyst loading using 75d as the
ligand for the diethylzinc addition to benzaldehyde

| entry | mol % of 75d | yield^{a} (%) | ee (R) (%) |
|---|---|---|---|
| 1 | 20 | 66 | 95 |
| 2 | 10 | 55 | 95 |
| 3 | 5 | 89 | 95 |
| 4 | 2 | 83 | 89 |

^{a}Lower yield due to difficulties in purification.

Using the optimized asymmetric reaction conditions, 75d was used to catalyze the addition of diethylzinc to other aldehyde substrates as shown in Table 5. The —Cl and —OCH₃ substituents on the aromatic ring did not seem to have a large effect on the enantiocontrol of the reaction (entries 2 and 3) and afforded results similar to benzaldehyde; however, the yield of the reaction was lower with chlorobenzaldehyde as the substrate. When cinnamaldehyde and hydrocinnamaldehyde were employed in the reaction, a decrease in the enantioselectivity was observed.

TABLE 5
Diethylzinc additions to aldehydes catalyzed by 75d (5 mol %)

| entry | aldehydes | yield (%) | ee (R) (%) |
|---|---|---|---|
| 1 | benzaldehyde | 89 | 95 |
| 2 | p-chlorobenzaldehyde | 57 | 94 |
| 3 | p-methoxybenzaldehyde | 78 | 94 |
| 4 | hydrocinnamaldehyde | 43 | 69 |
| 5 | cinnamaldehyde | 88 | 72 |

Overall, the novel nicotine-based decafluorodiphenyl catalyst demonstrates respectable yields and high enantioselectivities when employed in the catalytic asymmetric addition of diethylzinc to aromatic aldehydes.

Finally, to investigate the effect of the chlorine substituent on the nicotine-based catalysts during the asymmetric reaction, compounds 9028 and 91 were synthesized and compared to 75a (Schemes 3 and 4). This comparison would provide insight as to whether or not the C-4 substituted nicotine-based compounds could be improved to enhance selectivity in the asymmetric reaction by adding or removing chlorine substituents from the pyridine ring.

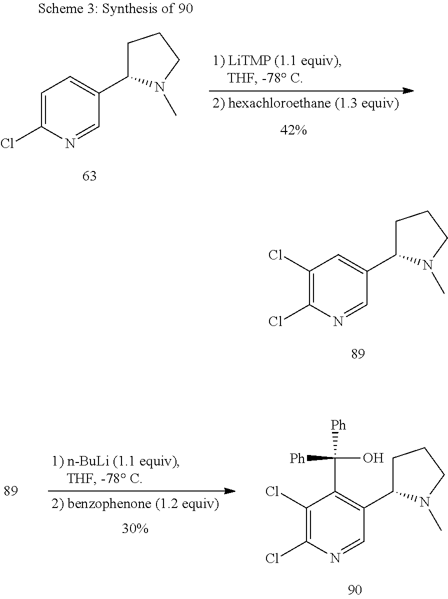

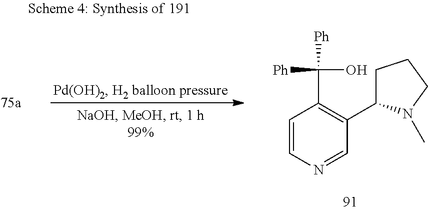

Addition of a chlorine substituent to the C-5 position of nicotine, as shown with compound 90, increased the selectivity to 95% ee at 20 mol %, but at 5 mol % a slight decrease in selectivity was observed (Table 6, entries 3 and 4). In contrast, catalyst 91 with no chlorine substituents decreased the selectivity as compared to ligand 75a as seen in Table 6, entries 5 and 6. The selectivity and yield were worse when the amount of catalyst 91 was lowered to 5 mol %. Therefore, it may be desirable to have at least one chlorine substituent on the nicotine portion of the molecule, in addition to the alcohol functionality off of the C-4 position, in order to maintain adequate selectivity in the asymmetric reaction.

TABLE 6
Comparing catalysts 75a, 75d, 90 and 91 in the asymmetric
reaction of diethylzinc and benzaldehyde^{a}

| entry | catalyst | mol % of cat. | yield (%) | ee (R) (%) |
|---|---|---|---|---|
| 1 | 75a | 20 | 78 | 79 |
| 2 | 75d | 5 | 89 | 95 |
| 3 | 90 | 20 | 61 | 95 |
| 4 | 90 | 5 | 91 | 92 |
| 5 | 91 | 20 | 69 | 63 |
| 6 | 91 | 5 | 36 | 60 |

^{a}Reactions were run at 0° C. for 24 h in toluene.

In conclusion, the more sterically hindered C-4 tertiary alcohols of (S)-6-chloronicotine provided better enantioselectivity than the C-4 secondary alcohols. Additionally, electron withdrawing fluorines on the aromatic ring improved the ee as compared to electron donating groups, as seen when comparing catalyst 75d with 75a and 75c. The effect of the chlorines on the pyridine ring of nicotine also proved to be beneficial when applied to the asymmetric reaction. Two chlorine substituents on nicotine derivative 90 proved to be superior to only one chlorine substituent on molecule 75a. When no chlorines were on C-4 substituted 91, the yield and the selectivity decreased noticeably.

Overall, compound 75d provided the most promising results, yielding an ee of 95% when benzaldehyde was the substrate and only 5 mol % of the catalyst was used. The catalyst also provided acceptable results when other substrates were employed in the asymmetric reaction.

However, compound 75d with zinc(II) triflate yielded very little product was obtained (data not shown). The diethylzinc conditions were applied where the product 93 was obtained with a 93% yield with a 13% ee, but some ethylphenyl alcohol 17 was also observed. In addition, when applied to the Henry reaction, the yields were very low (data not shown).

### Example 3

### Synthesis of C-4 Phosphine Ligands from (S)-6-Chloronicotine

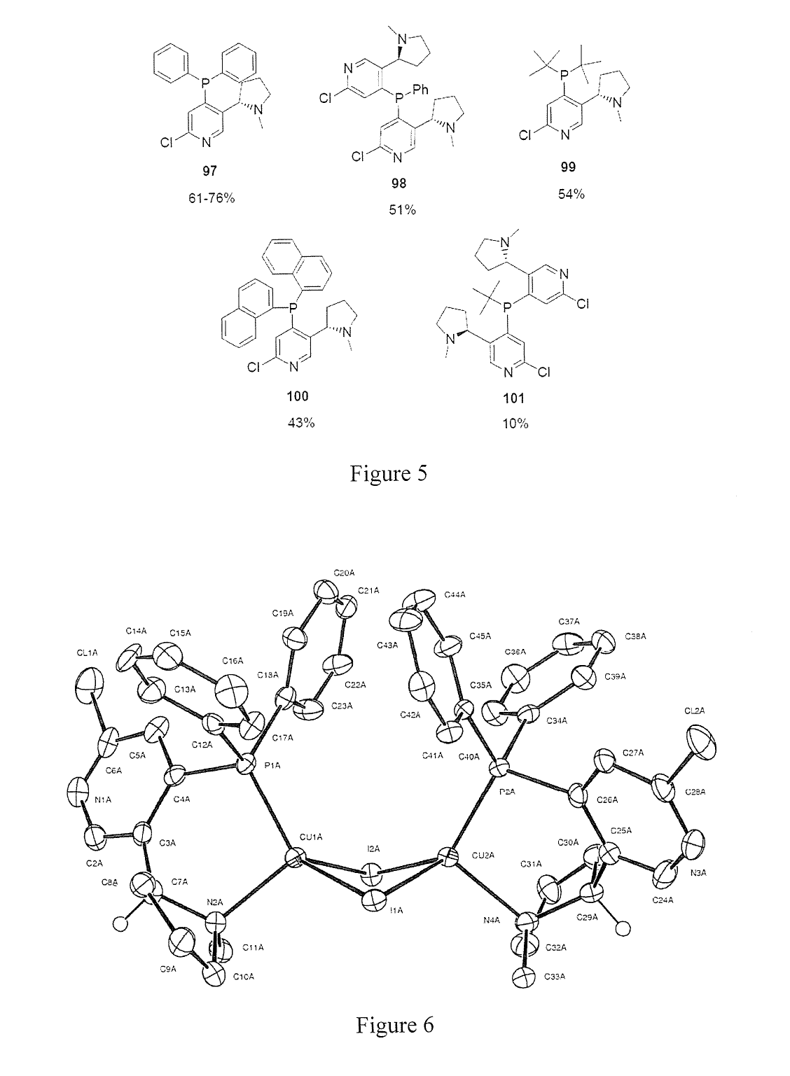

Chiral amino phosphines find wide application in asymmetric synthesis reactions, such as rhodium-catalyzed hydrogenations,47 1,4-conjugate additions,48 and palladium-catalyzed allylic alkylations.49 Phosphine catalysts were synthesized from (S)-6-chloronicotine using the C-4 lithiation procedure described above in combination with commercially available chloro- or dichlorophosphine electrophiles (see Table 9). Through this method, compounds 97, 98, 99, 100, and 101 were obtained with yields ranging from 10-76% (FIG. 5).

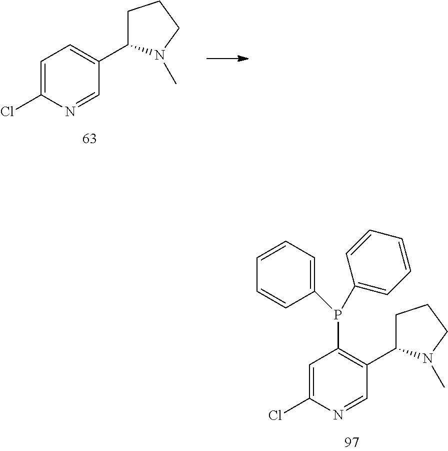

[5-((2S)-1-Methylpyrridin-2-yl)-2-chloro(4-pyridyl)]diphenylphosphine (97). To a solution of n-BuLi (0.47 mL, 2.30 M in hexanes) in THF (2 mL) was added (S)-6-chloronicotine (63, 194 mg, 0.99 mmol) at −78° C. The solution was stirred at −78° C. for 1 h followed by the dropwise addition of chlorodiphenylphosphine (0.21 mL, 1.18 mmol). The solution was stirred at −78° C. for 3 h then allowed to warm to near room temperature over 2 h. The reaction was then quenched with methanol, filtered, and concentrated. The crude mixture was purified by RPLC (1% TEA/20% EtOAc/hexanes) to afford 285 mg (76%) of 97 as a white solid, mp 116-118° C.; [α]30 D −109.2 (c 1.20, CH2Cl2); IR (thin film) 3069, 3055, 2966, 2939, 2837, 2782, 1562, 1435, 1363, 1311, 1119, 744 cm−1; 1H NMR (400 MHz, CDCl3) δ 8.59-8.58 (d, J=4.0 Hz, 1H); 7.38-7.37 (m, 6H), 7.28-7.23 (m, 4H), 6.65-6.64 (d, J=2.8 Hz, 1H), 3.66-3.60 (m, 1H), 3.14-3.10 (m, 1H), 2.20-2.13 (m, 1H), 1.97 (s, 3H), 1.87-1.74 (m, 2H), 1.68-1.59 (m, 1H), 1.49-1.41 (m, 1H); 13C NMR (75 MHz, CDCl3) δ 151.3, 150.9, 150.2, 149.42, 149.36, 141.9, 141.6, 134.8, 134.7, 134.5, 134.43, 134.38, 134.2, 129.85, 129.77, 129.2, 129.0, 126.5, 66.3, 66.1, 56.8, 40.2, 34.8, 23.1; 31P {1H} NMR (162 MHz, CDCl3) 6 −16.3; HRMS calcd for C22H22ClN2P ([M+H]+) 381.1281, found 381.1284.

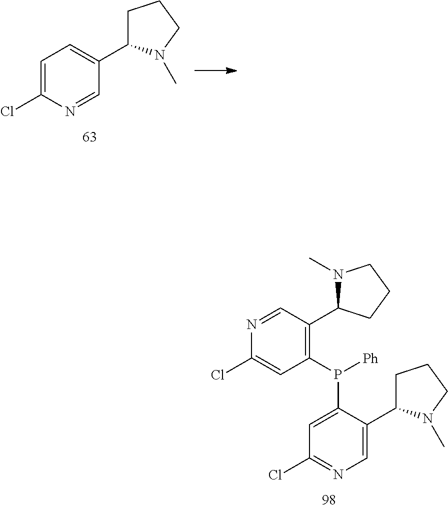

(S)-4,4′-(Phenylphosphinediyl)bis(2-chloro-5-((S)-methylpyrrolidin-2-yl)pyridine) (98). To a solution of n-BuLi (0.76 mL, 2.22 M in hexanes) in THF (3 mL) was added (S)-6-chloronicotine (63, 304 mg, 1.54 mmol) at −78° C. The solution was stirred at −78° C. for 1 h followed by the dropwise addition of dichlorophenylphosphine (0.13 mL, 0.93 mmol). The solution was stirred at −78° C. for 5 h then quenched with methanol, filtered and concentrated. The crude mixture was purified by RPLC (1% TEA/30% EtOAc/hexanes) to afford 195 mg (51%) of 98 as a white solid, mp 165-167° C.; [α]27 D −255.5 (c 1.75, CH2Cl2); IR (thin film) 3070, 3050, 2967, 2872, 2838, 2941, 2780, 1562, 1446, 1119 cm−1; 1H NMR (400 MHz, CDCl3) δ 8.57-8.54 (m, 2H), 7.47-7.39 (m, 3H), 7.26-7.21 (m, 2H), 6.63-6.59 (m, 2H), 3.65-3.60 (m, 1H), 3.46-3.41 (m, 1H), 3.17-3.08 (m, 2H), 2.25-2.18 (m, 1H), 2.15-2.08 (m, 1H), 2.01-1.87 (m, 8H), 1.85-1.78 (m, 1H), 1.75-1.63 (m, 4H), 1.60-1.51 (m, 1H); 13C NMR (100 MHz, CDCl3) δ 150.6, 150.29, 150.0, 149.9, 149.8, 149.7, 149.4, 148.6, 148.4, 141.5, 141.3, 141.2, 141.1, 134.9, 134.7, 132.4, 132.3, 130.6, 129.5, 129.4, 128.0, 127.3, 67.0, 66.9, 66.8, 66.7, 57.2, 56.7, 40.7, 40.3, 34.7, 34.6, 23.4, 23.4; 31P {1H} NMR (162 MHz, CDCl3) δ 29.2; HRMS calcd for C26H29Cl2N4P (M+) 499.1575, found 499.1579.

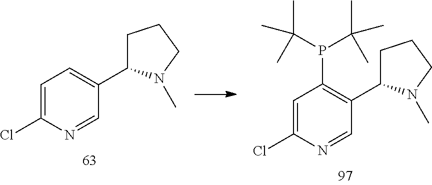

[5-((2S)-1-Methylpyrrolidin-2-yl)-2-chloro(4-pyridyl)]bis(tert-butyl)phosphine (99). To a solution of n-BuLi (0.24 mL, 2.22 M in hexanes) in THF (2 mL) was added (S)-6-chloronicotine (63, 97 mg, 0.54 mmol) at −78° C. The solution was stirred at −78° C. for 1 h followed by the dropwise addition of tert-butylchlorophosphine (0.11 mL, 0.59 mmol). The solution was stirred at −78° C. for 5 h and then slowly allowed to warm up to near room temperature over 2 h. The reaction was then quenched with methanol, filtered, and concentrated. The crude mixture was purified by RPLC (1% TEA/20% EtOAc/hexanes) to afford 91 mg, (54%) of 99 as a white solid, mp 125-127° C.; {a}31D −192.6 (c=1.30, CH2Cl2); IR (thin film) 2950, 2865, 2783, 1562, 1511, 1459, 1360, 1176, 1113, 1043, 740 cm−1; 1H NMR (400 MHz, CDCl3) δ 8.67-8.66 (d, J=4.0 Hz, 1H), 7.53 (s, 1H), 4.26-4.19 (m, 1H), 3.20-3.15 (m, 1H), 2.35-2.26 (m, 2H), 2.12 (s, 3H). 1.95-1.83 (m, 1H), 1.82-1.71 (m, 1H), 1.50-1.40 (m, 1H), 1.25-1.14 (m, 18H); 13C NMR (100 MHz, CDCl3) δ 150.1, 150.0, 149.5, 149.2, 148.2, 145.3, 145.1, 128.4, 128.3, 66.3, 66.1, 57.0, 40.6, 35.5, 33.4, 33.2, 32.5, 32.3, 30.8, 30.7, 30.4, 30.5, 22.9; 31P {1H}NMR (162 MHz, CDCl3) δ 11.9; HRMS calcd for C18H30ClN2P ([M+H]+) 341.1907, found 341.1907.

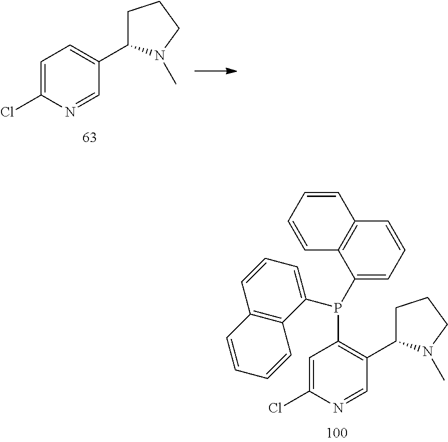

(S)-2-(4-Chloro-2-(dinaphthalen-1-ylphosphino)phenyl)-1-methylpyrrolidine (100). To a solution of n-BuLi (0.23 mL, 0.54 mmol) in THF (1 mL) at −78° C. was added (S)-6-chloronicotine (63, 97 mg, 0.49 mmol). After 1 h, a solution of chlorodi(1-naphthyl)phosphine (206 mg, 0.64 mmol) in THF (2 mL) at −78° C. was cannulated into the reaction and stirred for another 3 h. The reaction was slowly warmed up to −20° C. over 1 h, then stirred for an additional 2 h before diluting with methanol. The mixture was filtered and concentrated. Purification by RPLC (1% TEA/10% EtOAc/hexanes) was done to afford 108 mg (43%) of 100 as a white solid, mp 224-226° C.; [α]32 D −94.1 (c 1.25, CH2Cl2); IR (thin film, NaCl) 3052, 2966, 2940, 2838, 2785, 1562, 1503, 1443, 1362, 1119, 774 cm−1; 1H NMR (400 MHz, CDCl3) δ 8.69-8.68 (d, J=4.8 Hz, 1H), 8.46-8.43 (m, 1H), 8.37-8.34 (m, 1H), 7.93-7.89 (m, 4H), 7.56-7.47 (m, 4H), 7.39-7.29 (m, 2H), 7.01-6.96 (m, 2H), 6.62-6.61 (d, J=2.8 Hz, 1H), 3.81-3.75 (m, 1H), 3.12-3.08 (m, 1H), 2.16-2.04 (m, 4H), 1.96-1.87 (m, 2H), 1.72-1.59 (m, 2H); 13C NMR (75 MHz, CDCl3) δ 150.3, 150.0, 149.7, 149.4, 149.3, 142.4, 142.2, 135.8, 135.7, 135.4, 135.4, 134.2, 133.9, 133.8, 133.7, 133.5, 131.3, 131.2, 131.1, 130.7, 130.6, 129.2, 129.1, 129.0, 127.8, 126.9, 126.6, 126.5, 126.2, 126.0, 125.9, 125.8, 125.7, 66.7, 66.4, 56.8, 40.4, 34.9, 23.2; 31P {1H} NMR (162 MHz, CDCl3) δ 27.2; HRMS calcd for C30H26ClN2P (M+) 480.1522, found 480.1517.

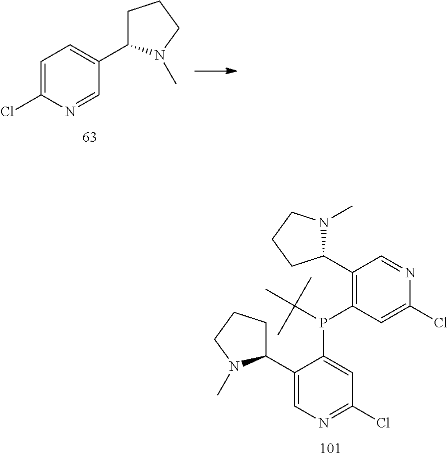

(S)-4,4′-(tert-Butylphosphinediyl)bis(2-chloro-5-((S)-1-methylpyrrolidin-2-yl)pyridine) (101). To a solution of n-BuLi (0.67 mL, 1.56 mmol) in THF (3 mL) at −78° C. was added (S)-6-chloronicotine (63, 306 mg, 1.56 mmol). After 1 h, a solution of tell-butyldichlorophosphine (118 mg, 742 mmol) in THF (1 mL) was added at −78° C. and stirred for 4 h before slowly warming up to −20° C. over 1 h. After stirring at −20° C. for 1 h, the reaction was then kept in a −20° C. refrigerator for 18 h before quenching with saturated sodium bicarbonate (5 mL) and extracting with methylene chloride (3×10 mL). The combined organic layers were dried over potassium carbonate, filtered, and concentrated. Purification by RPLC (1% TEA/2% EtOAc/hexanes) afforded 37 mg (10%) of 101 as a white sold, mp 58-60° C.; [α]35D −211.8 (c 1.80, CH2Cl2); IR (thin film, NaCl) 2967, 2941, 2870, 2838, 2781, 1561, 1444, 1359, 1118 cm−1; 1H NMR (400 MHz, CDCl3) δ 8.70-8.67 (dd, J=7.6 Hz, J=4.4 Hz, 2H), 7.19-7.17 (dd, J=5.2 Hz, J=2.0 Hz, 2H), 3.72-3.67 (m, 1H), 3.57-3.52 (m, 1H), 3.19-3.13 (m, 2H), 2.41-2.32 (m, 1H), 2.24-2.14 (m, 5H), 2.00-1.84 (m, 5H), 1.70-1.53, 3H), 1.45-1.27 (m, 11H); 13C NMR (75 MHz, CDCl3) δ 150.7, 150.6, 150.5, 150.4, 149.7, 149.6, 148.9, 148.7, 148.5, 148.3, 142.8, 142.7, 142.5, 142.4, 127.7, 127.3, 66.2, 65.9, 65.7, 65.5, 56.8, 40.3, 40.2, 35.6, 35.0, 31.9, 31.6, 28.9, 28.7, 23.1, 22.9; 31P {1H} NMR (162 MHz, CDCl3) 8 −10.0; HRMS calcd for C24H33Cl2N4P (M+) 478.1820, found 478.1811.

### Example 4

### Application of Chiral Amino Phosphine Ligands to Asymmetric Reactions

Phosphine 97 was examined in the catalytic asymmetric addition of diethylzinc to benzaldehyde using the same conditions described above. For both conditions attempted, both the yield and the ee were poor (data not shown). Phosphines 98 and 99 were not tested.

Phosphine compounds 97-101 were tested for the enantioselective conjugate addition of 1,4-dihydropyridones. Complex 115 was formed from copper(I) iodide and ligand 97 in toluene at room temperature (Scheme 5).

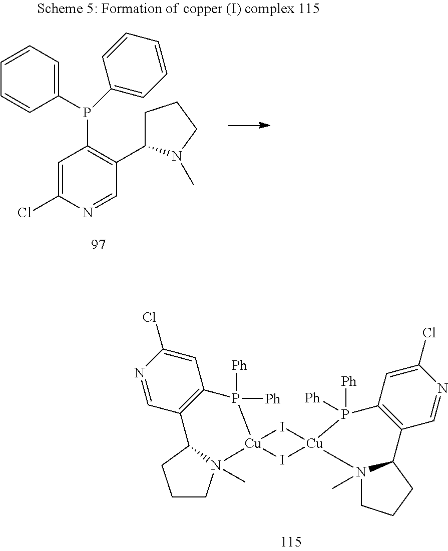

Copper iodide complex (115). To a solution of CuI (10 mg, 0.05 mmol) in toluene (0.2 mL) was added a solution of the phosphine ligand (97, 20 mg, 0.05 mmol) in toluene (0.2 mL) at room temperature. The reaction was stirred at room temperature for 1 h, and concentrated. The complex was isolated as a yellow solid (90% by 1H NMR). A single crystal of the complex was grown in an NMR tube with methylene chloride by slow evaporation under a nitrogen atmosphere. IR (thin film) 3188, 3052, 2961, 2853, 2791, 1561, 1450, 1436, 1125 cm−1; 1H NMR (300 MHz, CDCl3) δ 8.32-8.31 (d, J=4.2 Hz, 1H), 7.56-7.31 (m, 10H), 6.85-6.83 (d, J=7.2 Hz, 1H), 3.68-3.61 (m, 1H), 3.48-3.41 (m, 1H), 2.38-2.23 (m, 4H), 2.18-1.95 (m, 1H), 1.82-1.51 (m, 3H); 13C NMR (100 MHz, CDCl3) δ 152.3, 152.2, 134.6, 134.4, 134.3, 131.0, 130.4, 129.3, 129.2, 129.0, 128.9, 128.7, 72.9, 61.0, 47.0, 32.4, 22.2; 31P {1H} NMR (162 MHz, CDCl3) δ −23.3; HRMS calcd for C44H44Cl2Cu2I2N4P2 ([M+H]+) 1140.9172, found 1140.9143.

A single crystal of 115, used for structural elucidation by x-ray diffraction, was isolated by slow evaporation from methylene chloride in an inert atmosphere (FIG. 6). An attempt to grow a single crystal of the copper(II) triflate-98 complex was unsuccessful. However, the phosphines did not catalyze the reaction as only starting material was recovered (data not shown).

The chiral phosphines 97-101 were tested for the Pd-catalyzed asymmetric allylic alkylation reaction.

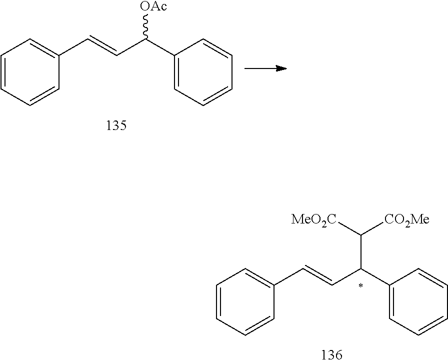

(S)-1,3-Diphenyl-1-dimethylmalonylprop-2-ene (136). Solution A: In a flask containing [Pd(η3-C3H5) Cl]2 (4 mg, 0.01 mmol) and 98 (10 mg, 0.02 mmol) was added methylene chloride (1 mL). The reaction was degassed for 10 min. After stirring at room temperature for 1 h, the solution was cooled to 0° C. where a solution of (rac)-1,3-diphenylallyl acetate 78 (135, 50 mg, 0.20 mmol) in methylene chloride (0.5 mL) over sieves was added by syringe. After a few minutes at 0° C., this solution was cannulated into solution B, which was at room temperature. Solution B: To a flask containing potassium carbonate (3 mg, 0.02 mmol) was added methylene chloride (1 mL), dimethylmalonate (0.07 mL, 0.59 mmol), and N,Obis(trimethylsilyl)acetamide (0.14 mL, 0.59 mmol) at room temperature. After 1 h at room temperature, solution A was cannulated into solution B. After the addition was complete, the reaction was stirred at room temperature for 19 h. The reaction was quenched with an aqueous saturated solution of ammonium chloride (1 mL), followed by extraction with methylene chloride (2×5 mL). The combined organic layers were dried over magnesium sulfate, filtered, and concentrated. Purification by RPLC (2% EtOAc/hexanes) afforded 45 mg (70%) of 136 as a colorless oil; 71% ee determined by HPLC analysis (Chiralcel AD column eluted with 10% i-propanol/hexanes at 1.00 mL/min and detection at λ=219-221 nm). Retention times: R (minor) 8.89 min, S (major) 11.74 min. 71,72,79

First, racemic 136 was synthesized using triphenylphosphine as a ligand to generate a reference sample for HPLC (Table 7, entry 1). Then, the catalysts 97-99 were tried under conditions where diethylzinc was utilized as a base (Table 7, entries 2-6). As shown in entries 5 and 6, both refluxing and stirring at room temperature in THF did not seem to affect the yield or the selectivity; therefore, reflux was often maintained so that the reaction would be completed in less time. However, when diethylzinc was employed as the base, foil nation of the reduced product 139 was obtained.

TABLE 7
Conditions for the asymmetric reaction using nicotine-based

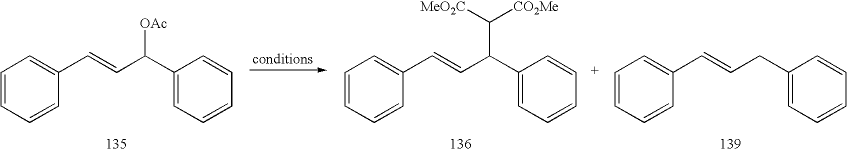

| entry | conditions | 136 (%) | ee (%)^{a} | | 139 (%) |
|---|---|---|---|---|---|
| 1 | [Pd(η³-C₃H₅)Cl]₂ (0.02 equiv), PPh₃(0.08 equiv), | 83 | 1 | | — |
| | dimethylmalonate (2.0 equiv), Et₂Zn (2.0 equiv), | | | | |
| | CH₂Cl₂, rt, 5 h | | | | |
| 2 | [Pd(η³-C₃H₅)Cl]₂ (0.02 equiv), ligand 97 (0.08 | 16 | 60 | (S) | 60 |
| | equiv), dimethylmalonate (2.0 equiv), Et₂Zn (2.0 | | | | |
| | equiv), THF, reflux, 5 h | | | | |
| 3 | [Pd(η³-C₃H₅)Cl]₂ (0.02 equiv), ligand 97 (0.08 | 8 | 63 | (S) | 80 |
| | equiv), dimethylmalonate (2.0 equiv), Et₂Zn (2.0 | | | | |
| | equiv), THF, reflux, 2 h | | | | |
| 4 | [Pd(η³-C₃H₅)Cl]₂ (0.02 equiv), ligand 99 (0.08 | 62 | 62 | (R) | 26 |
| | equiv), dimethylmalonate (2.0 equiv), Et₂Zn (2.0 | | | | |
| | equiv), THF, reflux, 1.5 h | | | | |
| 5 | [Pd(η³-C₃H₅)Cl]₂ (0.02 equiv), ligand 98 (0.08 | 64 | 68 | (S) | 28 |
| | equiv), dimethylmalonate (2.0 equiv), Et₂Zn (2.0 | | | | |
| | equiv), THF, reflux, 1.5 h | | | | |
| 6 | [Pd(η³-C₃H₅)Cl]₂ (0.02 equiv), ligand 98 (0.08 | 62 | 68 | (S) | 19 |
| | equiv), dimethylmalonate (2.0 equiv), Et₂Zn (2.0 | | | | |
| | equiv), THF, rt, 22 h | | | | |
| 7 | [Pd(η³-C₃H₅)Cl]₂ (0.05 equiv), ligand 98 (0.10 | 70 | 71 | (S) | — |
| | equiv), dimethylmalonate (3.0 equiv), BSA (3.0 | | | | |
| | equiv), CH₂Cl₂, rt, 19 h | | | | |
| 8 | [Pd(η³-C₃H₅)Cl]₂ (0.05 equiv), ligand 97 (0.10 | 14 | 9 | (S) | — |
| | equiv), dimethylmalonate (3.0 equiv), BSA (3.0 | | | | |
| | equiv), CH₂Cl₂, rt, 19 h | | | | |
| 9 | [Pd(η³-C₃H₅)Cl]₂ (0.05 equiv), ligand 100 (0.10 | 66 | 3 | (R) | — |
| | equiv), dimethylmalonate (3.0 equiv), BSA (3.0 | | | | |
| | equiv), CH₂Cl₂, rt, 19 h | | | | |
| 10 | [Pd(η³-C₃H₅)Cl]₂ (0.05 equiv), ligand 101 (0.10 | 67 | 43 | (R) | — |

equiv), dimethylmalonate (3.0 equiv), BSA (3.0
equiv), CH₂Cl₂, rt, 19 h

^{a}Determined by chiral HPLC on column Chiralpak AD with λ = 219-221 nm, 10% i-propanol/hexanes as the eluent with a flow rate of 1.00 mL/min.⁷⁴

To avoid the formation of 139 and attempt to increase the enantioselectivity, BSA was used as the base with catalysts 97, 98, 100, and 101 (Table 7, entries 7 and 10). Switching to these conditions with catalyst 97 proved to decrease the ee from 63% to 9% (entries 3 and 8). However, the enantioselectivity increased slightly from 68% to 71% for catalyst 98, which proved to be the best catalyst overall. Since ligand 100 provided the lowest selectivity, an additional tridentate ligand 101 was examined. Upon comparing results obtained from ligands 98 and 101, it might be the case that the tert-butyl group is detrimental to the selectivity of the reaction (entries 7 and 10). Overall, the performance of phosphines 98, 99, and 101 provided the best results in this reaction.

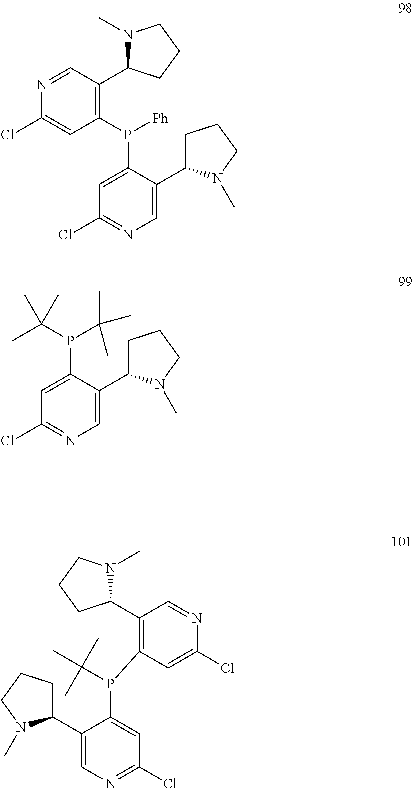

The foregoing is illustrative of the present invention, and is not to be construed as limiting thereof. The invention is defined by the following claims, with equivalents of the claims to be included therein.

1. A compound of Formula I: 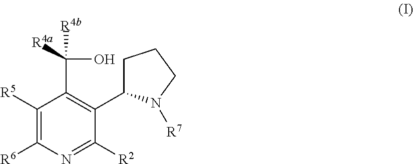
wherein:
R⁷ is H or alkyl;
R², R⁵ and R⁶ are each independently H, alkyl, halo, alkoxy, aryl or heteroaryl; and
R^{4a} and R^{4b} are each independently aryl or heteroaryl.

2. The compound of claim 1, wherein R², R⁵ and R⁶ are each independently H, alkyl, or halo.

3. The compound of claim 1, wherein R⁶ is chloro.

4. The compound of claim 1, wherein R^{4a} and R^{4b} are each independently aryl.

5. A compound of Formula (III)(A)(3)(a): 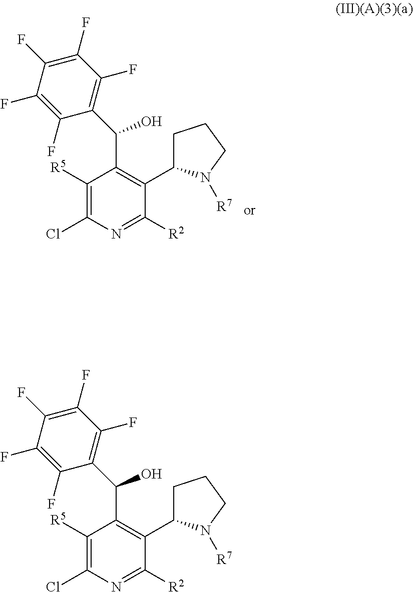
wherein:
R⁷ is H or alkyl; and
R² and R⁵ are each independently H, alkyl, halo, alkoxy, aryl or heteroaryl.

6. A compound of Formula II: 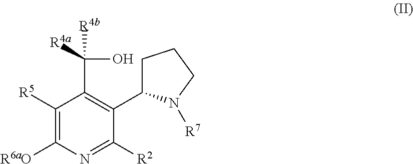
wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H, alkyl, aryl or heteroaryl.

7. A compound of Formula (III)(B)(4)(a): 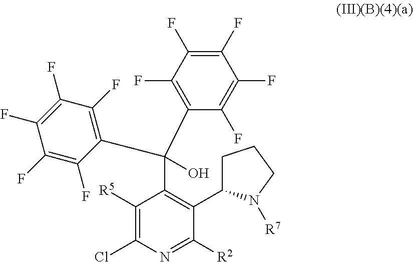
wherein:
R⁷ is H or alkyl; and
R² and R⁵ are each independently H, alkyl, halo, alkoxy, aryl or heteroaryl.

8. The compound of claim 7, wherein R² and R⁵ are each independently H, alkyl or halo.

9. A compound of Formula IV: 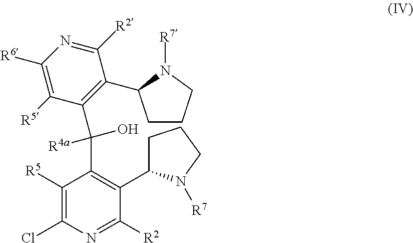
wherein:
R⁷ is H or alkyl;
R², R⁵ and R⁶ are each independently H, alkyl, halo, alkoxy, aryl or heteroaryl R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

10. The compound of claim 9, wherein R⁶ and R^{6′} are each independently halo.

11. The compound of claim 9, wherein R^{4a}, R², R^{2′}, R⁵ and R^{5′} are each H.

12. A method of catalytic asymmetric addition of a dialkylzinc to an aldehyde comprising catalyzing said asymmetric addition with a compound of claim 1.

13. A compound of claim 1 covalently bound to a solid support.

14. A complex comprising a metal and at least one compound of claim 1.

15. The complex of claim 14, wherein said metal is zinc.

16. A method of catalytic asymmetric addition of diethylzinc to an aldehyde comprising catalyzing said asymmetric addition with a complex of claim 14.

17. A compound of Formula X: 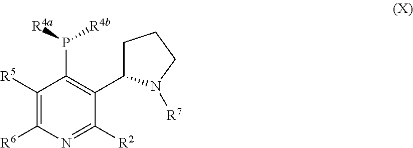
wherein:
R⁷ is H or alkyl;
R², R⁵ and R⁶ are each independently H, alkyl, halo, alkoxy, aryl or heteroaryl; and
R^{4a} and R^{4b} are each independently H, alkyl, aryl or heteroaryl.

18. The compound of claim 17, wherein R^{4a} and R^{4b} are each independently aryl or heteroaryl.

19. A compound Formula XI: 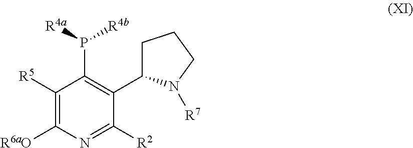
wherein:
R⁷ is H or alkyl;
R² and R⁵ are each independently H, alkyl, halo, aryl or heteroaryl;
R^{6a} is H, alkyl, aryl or heteroaryl; and
R^{4a} and R^{4b} are each independently H, alkyl, aryl or heteroaryl.

20. The compound of claim 19, wherein R^{4a} and R^{4b} are each independently aryl or heteroaryl.

21. A compound of Formula XII: 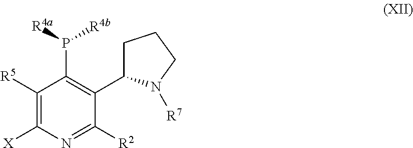
wherein:
R⁷ is H or alkyl;
R² and R⁵ are each independently H, alkyl, halo, alkoxy, aryl or heteroaryl;
X is halo; and
R^{4a} and R^{4b} are each independently H, alkyl, aryl or heteroaryl.

22. The compound of claim 21, wherein R^{4a} and R^{4b} are each independently aryl or heteroaryl.

23. The compound of claim 21, wherein X is chloro.

24. The compound of claim 21, wherein R^{4a} and R^{4b} are each phenyl.

25. A compound of Formula XIII: 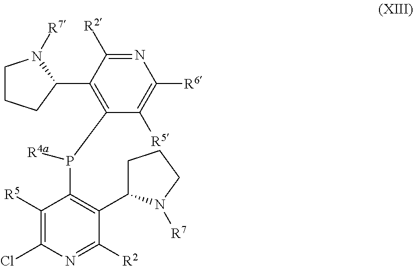
wherein:
R⁷ is H or alkyl;
R², R⁵ and R⁶ are each independently H, alkyl, halo, alkoxy, aryl or heteroaryl R^{4a} is H, alkyl, aryl or heteroaryl;
R^{2′}, R^{5′} and R^{6′} are each independently H, alkyl, halo, alkoxy, aryl or heteroaryl; and
R^{7′} is H or alkyl.

26. The compound of claim 25, wherein R², R⁵, R^{2′}, and R^{5′} are each H, and R⁶ and R^{6′} are each chloro.

27. A compound of claim 17 covalently bound to a solid support.

28. A complex comprising a metal and at least one compound of claim 17.

29. The complex of claim 28, wherein said metal is zinc.

30. The complex of claim 28, wherein said metal is copper.

31. A method of palladium-catalyzed allylic alkylation comprising catalyzing said allylic alkylation with a compound of claim 17.
